(19) United States
(12) Patent Application Publication
(10) Pub. No.: US 2025/0235496 A1
(43) Pub. Date: Jul. 24, 2025

(54) USE OF BELLIDIFOLIN (BEL) AND/OR BEL-CONTAINING EXTRACT IN PREPARATION OF ANTIDEPRESSANT PRODUCT

(71) Applicant: Northwest Institute of Plateau Biology, CAS, Qinghai (CN)

(72) Inventors: Yulin LI, Qinghai (CN); Tao CHEN, Qinghai (CN); Peipei LI, Qinghai (CN); Jing SUN, Qinghai (CN); Yumei MA, Qinghai (CN); Cheng SHEN, Qinghai (CN); Shuo WANG, Qinghai (CN)

(73) Assignee: Northwest Institute of Plateau Biology, CAS, Qinghai (CN)

(21) Appl. No.: 18/991,967

(22) Filed: Dec. 23, 2024

(30) Foreign Application Priority Data
Jan. 24, 2024 (CN) 202410099527.8

(51) Int. Cl. A61K 36/51; A61K 9/00; A61K 9/20; A61K 9/48; A61K 9/50; A61K 31/352; A61P 25/24

(52) U.S. Cl. CPC A61K 36/51; A61K 9/0056; A61K 9/0095; A61K 9/2031; A61K 9/485; A61K 9/4858; A61K 9/4866; A61K 9/5036; A61K 31/352; A61P 25/24; A61K 2236/31; A61K 2236/331; A61K 2236/333; A61K 2236/37; A61K 2236/39; A61K 2236/53

(57) ABSTRACT

Use of bellidifolin (BEL) and/or a BEL-containing extract in preparation of an antidepressant product is provided, belonging to the technical field of medicine. The BEL or BEL-containing extract as an active ingredient can significantly improve a depressive behavior of mice, regulate concentrations of 5-hydroxytryptamine (HT) and corticosterone (CORT), protect nerve cells, and improve synaptic plasticity. Moreover, the BEL or BEL-containing extract can penetrate the blood-brain barrier (BBB) and has a significant effect on improving and treating depression. In addition, a method for preparing the BEL-containing extract has the advantages of simple operation, low cost, and desirable repeatability and stability. BEL in the extract has a concentration of up to 1.0% to 95.0%. After purifying the BEL-containing extract, the BEL has a high purity of up to 95.0% to 99.9%, and is easy to realize industrial production during the preparation.

### CROSS REFERENCE TO RELATED APPLICATION

This patent application claims the benefit and priority of Chinese Patent Application No. 202410099527.8 filed with the China National Intellectual Property Administration on Jan. 24, 2024, the disclosure of which is incorporated by reference herein in its entirety as part of the present application.

### TECHNICAL FIELD

Use of bellidifolin (BEL) and/or a BEL-containing extract in preparation of an antidepressant product is provided, belonging to the technical field of medicine.

### BACKGROUND

Depression is a common mental illness, with main symptoms of low mood, loss of pleasure, irritability, inattention, and abnormal appetite and sleep. Depression has received increasing attention due to difficulty in cure and easy relapse, and patients with severe depression generally have suicidal thoughts and behaviors.

At present, the drugs for treating depression are mainly chemical synthetic drugs, including tricyclic antidepressants, monoamine oxidase inhibitors (MAOIs), selective 5-hydroxytryptamine (HT) reuptake inhibitors (SSRIs), noradrenergic and specific serotonergic antidepressants (NaSSAs), and 5-HT & norepinephrine reuptake in hibitors (SNRIs). Chemical drugs such as the MAOIs, SSRIs, and SNRIs are widely used, but have certain side effects in clinical treatment, such as vomiting, diarrhea, and weight loss. Since antidepressants require long-term administration, it is extremely necessary to develop natural drugs for treating depression with relatively less toxicity.

Bellidifolin (BEL) is a natural tetraoxone compound, mainly distributed in the plants of genera Swertia, Gentianella, and Gentiana of the Gentianaceae family. Pharmacological experiments have shown that the BEL has the effects of decreasing blood sugar, anti-oxidation, antibacterial, antiviral, inhibiting cholinesterase and monoamine oxidase, protecting cardiovascular system, and inhibiting ischemic brain injury. Through searching domestic and foreign literature, there is no literature report on the antidepressant effect of BEL.

### SUMMARY

To remedy the deficiencies of the prior art, the objective of the present disclosure is providing use of BEL and/or a BEL-containing extract in preparation of an antidepressant product.

The present disclosure provides use of BEL and/or a BEL-containing extract in preparation of an antidepressant product.

Preferably, the antidepressant product includes 0.1% to 99.9% of the BEL by weight.

Preferably, the antidepressant product is selected from the group consisting of a drug, a health product, and a functional food.

Preferably, the BEL-containing extract includes 1.0% to 95.0% of the BEL by weight.

Preferably, a method for preparing the BEL-containing extract includes the following steps:
mixing a BEL-containing raw material and an ethanol solution with a volume concentration of 65% to 75% to allow heating reflux extraction, and separating ethanol from an obtained extract to obtain a BEL crude extract;
dissolving the BEL crude extract in hot water at not less than 90° C., and mixing an obtained solution with concentrated hydrochloric acid to allow hydrolysis to obtain an acid hydrolyzate;
adjusting a pH value of the acid hydrolyzate to 7 to allow salting out, and collecting an obtained first precipitate;
dissolving the precipitate by heating in an ethanol solution with a volume concentration of 95.0% or pure methanol, cooling, conducting solid-liquid separation, and collecting an obtained solution; and
mixing the solution with water, allowing an obtained mixed solution to stand, and collecting an obtained second precipitate to obtain the BEL-containing extract.

Preferably, the heating reflux extraction is conducted at not less than 90° C. for 1 to 3 times, 2 h each time.

Preferably, the solid-liquid ratio of BEL-containing raw material and the ethanol solution are 1 g: 20 mL to 1 g: 30 mL during each time of the heating reflux extraction; and
the BEL-containing raw material is selected from the group consisting of Swertia mussotii, Swertia franchetiana, Swertia chirayita, Lomatogonium carinthiacum, and Halenia elliptica.

Preferably, the hydrolysis is conducted at not less than 90° C. for 0.5 h to 1 h.

Preferably, the pH value of the acid hydrolyzate is adjusted using a sodium hydroxide solution;
the salting out is conducted with sodium chloride for not less than 2 h; and
the mixed solution is allowed to stand for not less than 24 h.

Preferably, after obtaining the BEL-containing extract, further purifying the BEL-containing extract by preparative liquid chromatography to obtain BEL with a purity of 95.0% to 99.9%.

### Beneficial Effects:

BEL is a natural tetraoxo ketone compound. In the present disclosure, the BEL or BEL-containing extract as an active ingredient can significantly improve a depressive behavior of mice, regulate concentrations of 5-hydroxytryptamine (HT) and corticosterone (CORT), protect nerve cells, and improve synaptic plasticity. Moreover, the BEL or BEL-containing extract can penetrate the blood-brain barrier (BBB) and has a significant effect on improving and treating depression and addressing the side effects of weight loss and diarrhea.

In addition, a method for preparing the BEL-containing extract has simple operation, low cost, and desirable repeatability and stability. BEL in the extract has a concentration of up to 1.0% to 95.0%. After purifying the BEL-containing extract, the BEL has a high purity of up to 95.0% to 99.9%, and is easy to realize industrial production during the preparation.

### BRIEF DESCRIPTION OF THE DRAWINGS

To illustrate the examples of the present disclosure or the technical solutions in the prior art more clearly, the drawings required in the examples will be briefly introduced below.

FIG. 1 shows a high-performance liquid chromatography (HPLC) map of Swertia mussotii;

FIG. 2 shows a HPLC analysis chart of the BEL-containing extract obtained in Example 2;

FIG. 3 shows a medium-pressure preparative liquid chromatography separation diagram of the BEL obtained in Example 2;

FIG. 4 shows a HPLC analysis chart of the BEL obtained in Example 2;

FIG. 5 shows the viability of HT22 cells induced by different concentrations of CORT; where * compared with the induced group, *: P<0.05, **: P<0.01;

FIG. 6 shows an influence from different concentrations of compounds on CORT-induced HT22 cell viability;

FIG. 7 shows weight changes of mice in a BEL acute toxicity experiment (n=5);

FIG. 8 shows an influence of the BEL on a brain coefficient of mice (n=5);

FIGS. 9A-C show influence of the BEL on mouse liver (n=5); where FIG. 9A is liver coefficient, FIG. 9B is aspartate transaminase (AST) index, FIG. 9C is alanine aminotransferase (ALT) index;

FIGS. 10A-C show influence of the BEL on mouse kidney (n=5); where FIG. 10A is kidney coefficient, FIG. 10B is blood urea nitrogen (BUN) index, FIG. 10C is creatinine (CRE) index;

FIG. 11 shows an experimental flow chart of in vivo activity verification of BEL;

FIGS. 12A-C show influence of the BEL on changes in (FIG. 12A) body weight, (FIG. 12B) diet, and (FIG. 12C) water intake of mice (n=10);

FIGS. 13A-D show results of a mouse behavioral test (n=10); where FIG. 13A is the immobility time in FST of mice in the normal group, model group, and BEL-treated group;

FIG. 13B is the immobility time in TST of mice in the normal group, model group, and BEL-treated group; FIG. 13C is the SPT sugar water preference of mice in the normal group, model group, and BEL-treated group; FIG. 13D is the start time of eating in NSFT of mice in the normal group, model group, and BEL-treated group; * compared with the blank group, **: P<0.01, #compared with the model group, #: P<0.05;

FIGS. 14A-B show influence of the BEL on serum 5-HT and CORT levels (n=10);

FIG. 15 shows an influence of the BEL on a structure of the hippocampus in mice;

FIG. 16 shows an influence of the BEL on proliferation of neurons in the hippocampus of mice (immunohistofluorescence, 200×);

FIG. 17 shows an influence of the BEL on proliferation of neurons in the hippocampus of mice (immunohistofluorescence intensity analysis, n=6);

FIG. 18 shows Golgi staining (100×) of brain tissues of mice in the normal group, model group, and BEL-treated group;

FIG. 19 shows an influence of the BEL on a dendritic spine density of hippocampal neurons (n=15);

FIGS. 20A-C show mass spectrometry imaging results of mouse brain tissue under the action of BEL; where FIG. 20A is the structure of mouse brain tissue; FIG. 20B is the mass spectrometry imaging of the targeted compound; FIG. 20C is the ion intensity of the targeted compound (n=3);

FIG. 21A to FIG. 21C show an influence of the BEL on a mass spectrometry signal of mouse brain; where FIG. 21A is a number of ions in the mouse brain in the normal group and the BEL-treated group; FIG. 21B is a heat map of ion expression abundance; FIG. 21C is an MS map of the mouse brain in the normal group and the BEL-treated group;

FIG. 22 shows analysis results of ion clustering in the mouse brain in the normal group and the BEL-treated group;

FIGS. 23A-D show influence of the BEL on the expression of p-CREB/CREB in the hippocampus of depressed mice (n=3); where FIG. 23A is the immunoblot band of CREB and p-CREB protein expression in the hippocampus of the normal group, model group and BEL-treated group; FIG. 23B is a bar graph of the relative expression level of p-CREB protein in the hippocampus of the normal group, model group, and BEL-treated group; FIG. 23C is a bar graph of the relative expression level of CREB protein in the hippocampus of the normal group, model group, and BEL-treated group; FIG. 23D is the phosphorylation degree of CREB in the hippocampus of the normal group, model group, and BEL-treated group;

FIGS. 24A-C show influence of the BEL on the expression of 5-HT receptor protein in the hippocampus of depressed mice (n=3); where FIG. 24A is the immunoblot band of the expression of 5-HT1A and 5-HT2A proteins in the normal group, model group, and BEL-treated group;

FIG. 24B is a bar graph of the relative expression level of 5-HT1A protein in the hippocampus in the normal group, model group, and BEL-treated group; FIG. 24C is a bar graph of the relative expression level of 5-HT2A protein in the hippocampus in the normal group, model group, and BEL-treated group;

FIGS. 25A-B show influence of the BEL on the expression of Arg1 protein in the hippocampus of depressed mice (n=3); where FIG. 25A is the immunoblot band of Arg1 protein expression in the hippocampus of the normal group, model group, and BEL-treated group;

FIG. 25B is a bar graph of the relative expression level of Arg1 protein in the hippocampus of the normal group, model group, and BEL-treated group;

FIG. 26 shows an influence from different concentrations of BEL extracts on CORT-induced HT22 cell viability; where * compared with the induced group, **: P<0.01;

FIGS. 27A-C show influence of the BEL extract on changes of body weight, diet, and water intake of mice during an administration period (n=10); where * compared with the blank group, **: P<0.01;

FIGS. 28A-D show influence of the BEL extract on mouse behavior (n=10); where FIG. 28A is the immobility time in FST of mice in the normal group, model group, and BEL extract-treated group; FIG. 28B is the immobility time in TST of mice in the normal group, model group, and BEL extract-treated group; FIG. 28C is the SPT sugar water preference of mice in the normal group, model group, and BEL extract-treated group; FIG. 28D is the start time of eating in NSFT of mice in the normal group, model group, and BEL extract-treated group; where * compared with the blank group, **: P<0.01, #compared with the model group, #: P<0.05, ##: P<0.01; and

FIGS. 29A-B show influence of the BEL extract on 5-HT and CORT levels in serum (n=10); where * compared with the blank group, *: P<0.05, **: P<0.01, #compared with the model group, #: P<0.05, ##: P<0.0.

### DETAILED DESCRIPTION OF THE EMBODIMENTS

The present disclosure provides use of BEL and/or a BEL-containing extract in preparation of an antidepressant product.

In the present disclosure, the antidepressant product includes preferably 0.1% to 99.9% of the BEL by weight. The BEL has a structural formula as follows:

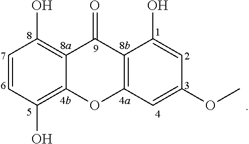 [SMILES: COc1cc(O)c2c(=O)c3c(O)ccc(O)c3oc2c1]

In the present disclosure, the antidepressant product is preferably selected from the group consisting of a drug, a health product, and a functional food. There is no strict requirement for a dosage form of the drug, the health product, or the functional food, and conventional selections may be used, such as solid beverages, candy tablets, capsules, microcapsules, and dripping pills.

In the present disclosure, the BEL-containing extract includes 1.0% to 95.0% of the BEL by weight.

In the present disclosure, a BEL-containing raw material and an ethanol solution with a volume concentration of 65% to 75% are mixed to allow heating reflux extraction, and ethanol is separated from an obtained extract to obtain a BEL crude extract. The heating reflux extraction is conducted at preferably not less than 90° C., more preferably 90° C. preferably 1 to 3 times, more preferably 2 times. Extracts obtained from each time of the heating reflux extraction are combined. The ethanol in the extract is preferably separated by vacuum concentration.

In the present disclosure, each time of the heating reflux extraction is conducted for preferably 2 h; the solid-liquid ratio of BEL-containing raw material and the ethanol solution are preferably 1 g: 20 mL to 1 g: 30 mL, more preferably 1 g: 25 mL. The BEL-containing raw material is preferably selected from the group consisting of Swertia mussotii, Swertia franchetiana, Swertia chirayita, Lomatogonium carinthiacum, and Halenia elliptica.

In the present disclosure, the BEL crude extract is dissolved in hot water at not less than 90° C., and an obtained solution is mixed with concentrated hydrochloric acid to allow hydrolysis to obtain an acid hydrolyzate. The hot water is preferably 90° C. A total volume of the hot water and the BEL crude extract is at a ratio of preferably 100:1 to a volume of the concentrated hydrochloric acid. The hydrolysis is conducted at preferably not less than 90° C., more preferably 90° C. The hydrolysis is conducted for preferably 0.5 h to 1 h, more preferably 0.8 h.

In the present disclosure, a pH value of the acid hydrolyzate is adjusted to 7 to allow salting out, and an obtained first precipitate is collected. The pH value of the acid hydrolyzate is preferably adjusted using sodium hydroxide solution. The salting out is preferably conducted by sodium chloride for preferably not less than 2 h. There is no strict requirement for a process of collecting the precipitate, and filtrate can be removed by centrifugation.

In the present disclosure, the precipitate is dissolved by heating in an ethanol solution with a volume concentration of 95.0% or methanol, cooled, then a solid-liquid separation is conducted, and an obtained solution is collected. The dissolving by heating is conducted at preferably 90° C. The solid-liquid separation is preferably conducted by filtration.

In the present disclosure, the obtained solution is mixed with water, an obtained mixed solution is allowed to stand, and an obtained second precipitate is collected to obtain the BEL-containing extract. The solution and the water are at a volume ratio of preferably 1:0.5 to 1:1; the mixed solution is allowed to stand for preferably not less than 24 h.

In the present disclosure, after obtaining the BEL-containing extract, preferably the BEL-containing extract is further purified by preparative liquid chromatography to obtain BEL with a purity of preferably 95.0% to 99.9%.

In the present disclosure, a CORT-induced HT22 neural cell model is used to screen out the antidepressant active monomer component BEL (C) and an active part comprising BEL-containing extract (C-TQW) by taking cell activity as an index. The pharmacodynamics of the antidepressant effect of BEL and BEL-containing extract are studied in the behavioral, histomorphological, and biochemical aspects of the mice using a CORT-induced Kunming mouse model. Finally, the antidepressant mechanism of BEL is explored based on in vivo experiments. The results show that the BEL or BEL-containing extract as an active ingredient can significantly improve a depressive behavior of mice, regulate concentrations of 5-HT and CORT, protect nerve cells, and improve synaptic plasticity. Moreover, the BEL or BEL-containing extract can penetrate the blood-brain barrier (BBB) and has a significant effect on improving and treating depression. Antidepressant products can be prepared by using BEL as the only active ingredient or the BEL-containing extract as the only active site, or a mixture of the BEL and the BEL-containing extract as active ingredients to improve and treat depression. In addition, a preparation method of the BEL-containing extract has simple processes, low cost, and desirable repeatability and stability. BEL in the extract has a concentration of up to 1.0% to 95.0%. After purifying the BEL-containing extract, the BEL has a high purity of up to 95.0% to 99.9%, and it is easy to realize industrial production during the preparation.

In order to further illustrate the present disclosure, the use of BEL and/or a BEL-containing extract in preparation of an antidepressant product provided by the present disclosure is described in detail below in connection with drawings and examples, but these examples should not be understood as limiting the claimed scope of the present disclosure.

In the present disclosure, the test instruments used include: XS204 electronic balance (Switzerland, Mettler-Toledo); TCS-150 electronic platform scale (Zhejiang Yuyang Weighing Apparatus Co., Ltd.); QJY-200 straight-cutting herb cutter (Shanghai Nanjue Herb Equipment Manufacturing Co., Ltd.); R-1020 rotary evaporator (Zhengzhou Greatwall Scientific Industrial and Trade Co., Ltd.); TQG-0.3-01 extraction tank (Zhejiang Wenxiong Mechanical Valve Industry Co., Ltd.); SJN-150-00 double-effect concentrator (Zhejiang Wenxiong Mechanical Valve Industry Co., Ltd.); F50H2 glass reactor (Gongyi Yingyu Rivet Factory); GQ75 high-speed tubular centrifuge separator (Shanghai Tianben Machinery Technology Co., Ltd.); DHG-9140A electric constant temperature blast drying oven (Shanghai Yiheng Technology Co., Ltd.); DAC-HB100 medium-pressure preparative liquid chromatography (Jiangsu Hanbon Science & Technology Co., Ltd.); LC3100 high-performance liquid chromatograph (Anhui Wanyi Science and Technology Co., Ltd.); SYH laboratory three-dimensional motion mixer (Jiangsu Chitong Machinery Manufacturing Co., Ltd.); Encapsulator B-390 microcapsule granulator (BUCHI); GL2-25 laboratory dry granulator (Jiangsu Zhangjiagang Kaichuang Machinery Manufacturing Co., Ltd.); NJP-2-200C fully automatic capsule filling machine (Ruian Tianhong Pharmaceutical Machinery Co., Ltd.); ZPSX10 rotary tablet press (Shanghai Xiangshun Pharmaceutical Machinery Co., Ltd.); GLZY-0.5B freeze dryer (Shanghai Pudong Freeze Drying Equipment Co., Ltd.); DW-1 small pill dropping machine (Changsha Yiguang Pharmaceutical Machinery Co., Ltd.).

In the present disclosure, the reagents and materials used include: methanol (for HPLC analysis, chromatographically pure) (Shandong Yuwang Company); preparative chromatography methanol (analytically pure) (Chengdu Kelong Chemical Co., Ltd.); ethanol (pharmaceutical grade) (Qinghai Huzhu Tianyoude Highland Barley Spirits Co., Ltd.); concentrated hydrochloric acid (analytically pure) (Sichuan Xilong Scientific Co., Ltd.); sodium hydroxide (analytically pure) (Tianjin Kaitong Chemical Reagent Co., Ltd.); sodium chloride is industrial salt (Golmud Baojin Chemical Co., Ltd.); and experimental water (pure water and ultrapure water). Whole herb of Swertia mussotii and Swertia franchetiana are provided by Northwest Plateau Institute of Biology, Chinese Academy of Sciences.

In the present disclosure, the efficacy test instruments used include: CO₂ incubator (HERACELL 150i CO2 Incubator, Thermo Scientific, USA), decolorization shaker (WD-9405F, Beijing Liuyi Biotechnology Co., Ltd., China), upright microscope (Nikon ECLIPSE Ts 2, Nikon, Japan), water bath (XMTE-8112, Jinghong, China), low-speed centrifuge (L500-A, Xiangyi, China), freezing microtome (CM 1860 UV, Leica, Germany), low-temperature high-speed centrifuge (Centrifuge 5424 R, Eppendorf, Germany), microplate reader (MULTISKAN FC, Thermo Scientific, USA), fluorescence inverted microscope (DS-Ri2, Nikon Eclipse, Japan), mini centrifuge (WTL, Changsha Xiangyi Centrifuge Instrument Co., Ltd., China), incubator (HB 120-S, DWB, China), decolorization shaker (WD-9405F, Beijing Liuyi Biotechnology Co., Ltd., China), fully automatic chemiluminescence image analysis system (Tanon 5200, Shanghai Tanon Life Science Co., Ltd., China), High speed refrigerated centrifuge (Centrifuge 5424 R(502), Eppendorf, Germany), water purifier (Millipore-Q Integral 3, Merk, Germany), confocal microscope (Leica DFC 7000T, Leica, Japan), SDS-polyacrylamide electrophoresis system (Bio Rad, USA), fully automatic chemiluminescence image analysis system (Tanon 5200, Shanghai Tanon Life Science Co., Ltd.), MALDI-TOF/TOF mass spectrometer (Bruker, Germany).

In the present disclosure, the drug efficacy test reagents and animals used include: duloxetine hydrochloride (D808419, Macklin), fluoxetine hydrochloride (F131623, Aladdin), imipramine hydrochloride (113-52-0, Macklin), venlafaxine (V873648, Macklin), BEL (11-3720-2020, self-made by the Northwest Institute of Plateau Biology, Chinese Academy of Sciences), 3-(4,5-dimethylthiazol-2)-2,5-diphenyltetrazolium bromide (MTT, M5655, Sigma), fetal bovine serum (FBS, 10099-141, Gibco), 0.25% trypsin (25200-056, Gibco), DMEM medium (C3113-0500, Gibco), CORT (C104537, Aladdin), dimethyl sulfoxide (C104537, Aladdin), hematoxylin-eosin stain (C0105S, Beyotime), mouse 5-hydroxytryptamine (5-HT) ELISA kit (H104-1-2, Nanjing Jiancheng), mouse corticosterone (CORT) ELISA kit (H205-1-2, Nanjing Jiancheng), Golgi kit (HTKNS1125NH, Hitobiotec Corp.), PBS dry powder (Coolaber), BSA Fraction V (ST023-200 g, Beyotime), Alexa Fluor 488-labeled goat anti-mouse IgG (H+L) (A0428, Beyotime), Cy3-labeled goat anti-rabbit IgG (H+L) (A0516, Beyotime), 2-MBT (M3302, Sigma), DMCA (D133809, Sigma), Brdu Antibody (ab8152, Abcam), Color-coded Prestained Protein Marker (#74124, Cell Signaling Technology), Phospho-CREB (#9198, Cell Signaling Technology), CREB (#9197, Cell Signaling Technology), iNOS (#13120, Cell Signaling Technology), Arginase-1 (#93668, Cell Signaling Technology), Anti-5-HT2A (abs146062, Absin), Anti-5-HT1A (abs133798, Absin), Anti-SLC6A4 (abs151709, Absin), β-actin (#4970, Cell Signaling Technology), TBSTw (10X) (ST673, Beyotime), 1.5 M Tris-HCl (pH=8.8) (ST789, Beyotime), 1 M Tris-HCl (pH=6.8) (ST768, Beyotime), SDS (3250GR500, BioFrox), glycine (1275GR500, BioFrox), Tris(1115GR500, BioFrox), ammonium persulfate (ST005, Beyotime), 30% Acr-Bis (29:1) (ST003, Beyotime), TEMED (ST728, Beyotime), and WB secondary antibody (SA00001-2, Wuhan Proteintech). Male Kunming (KM) mice are obtained from the Laboratory Animal Center, Xi'an Jiaotong University Health Science Center, with license number: SCXK 2018-001.

### Example 1

### 1. A Method for Preparing a BEL-Containing Extract Included the Following Steps:

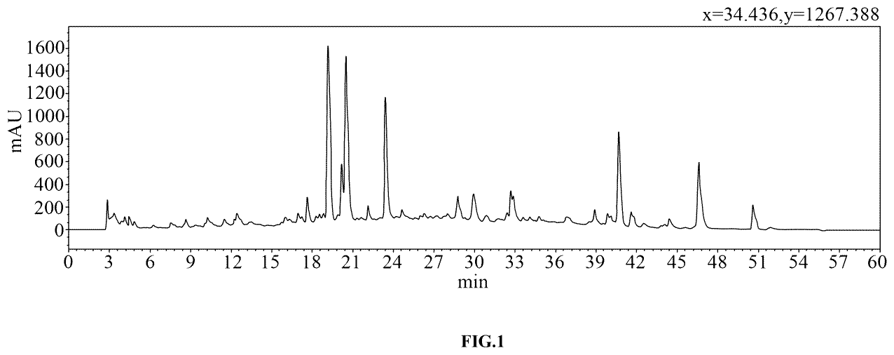

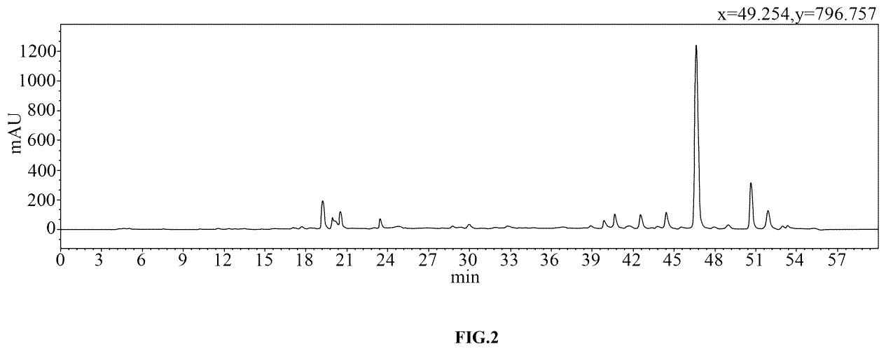

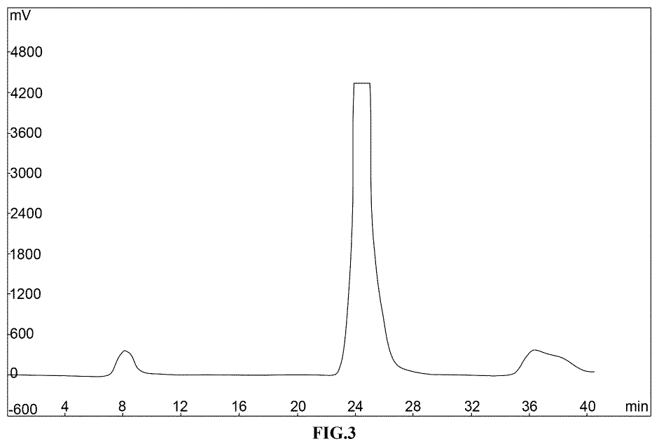

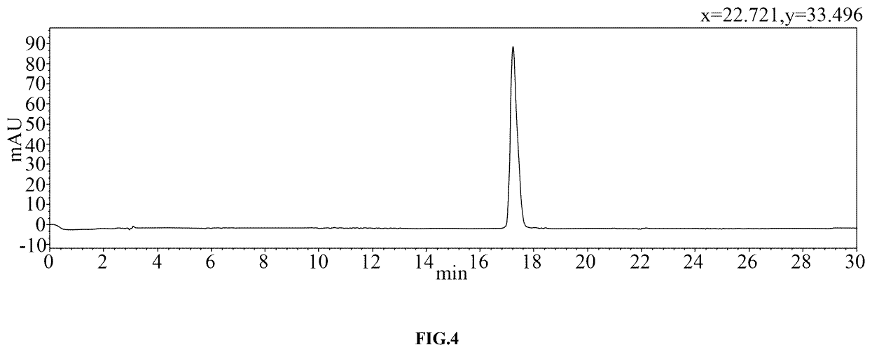

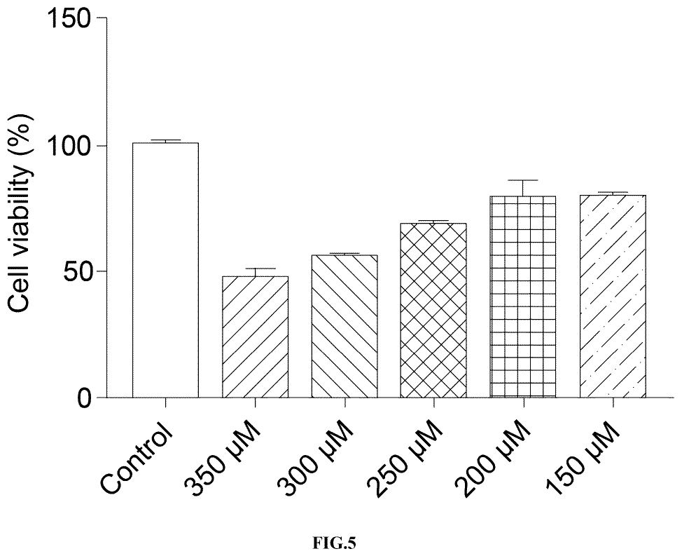

(1) Extraction: 10 kg of dried whole herb of Swertia mussotii (with HPLC map shown in FIG. 1) was chopped with a QJY-200 straight-cutting herb cutter, and added with 300 L of 70% ethanol solution at a solid-to-liquid ratio of 1 g: 30 mL. An obtained mixture was subjected to heating reflux extraction at 90° C. in a TQG-0.3-01 extraction tank for 3 times, each time for 2 h. Obtained extracts were combined and subjected to vacuum concentration in an SJN-150-00 double-effect concentrator to recover ethanol. The combined extracts were further concentrated in an R-1020 rotary evaporator to obtain 6.5 kg of a crude extract (with water content of 60%).
(2) Hot-melt and acid hydrolysis: the crude extract in step (1) was dissolved in 50 L of hot water at not less than 90° C. in a F50H2 glass reactor, added with 500 mL of concentrated hydrochloric acid and stirred evenly, and an obtained mixture was subjected to hydrolysis at 90° C. for 1 h to obtain an acid hydrolyzate.
(3)Alkali neutralization and salting out precipitation: the acid hydrolyzate in step (2) was neutralized with sodium hydroxide solution to a pH value of 7, then added with 3,000 g of sodium chloride and dissolved by stirring, and an obtained mixture was allowed to stand for 2 h to obtain a precipitate-containing solution.
(4) Centrifugation: the precipitate-containing solution in step (3) was centrifuged using a GQ75 high-speed tubular separator to remove the solution, and then washed with clean water 2 to 3 times to obtain a dark brown precipitate.
(5) Hot-melt extraction and recrystallization: the dark brown precipitate in step (4) was dissolved by heating with a small amount of ethanol solution at a volume concentration of 95.0% or pure methanol, cooled, filtered to obtain a filtrate, the filtrate was added with an equal volume of water, stirred evenly, then allowed to stand for not less than 24 h, and a yellow powder was precipitated. The yellow powder was filtered and dried to obtain 116 g of the BEL-containing extract. According to LC3100 HPLC analysis, the BEL-containing extract had a BEL content of 52.6% by weight.

### Preparative Liquid Chromatography Purification of the BEL

The BEL-containing extract obtained in step 1 was fully dissolved in methanol with a volume concentration of 85% and then separated and purified using a DAC-HB100 preparative liquid chromatograph. Methanol with a volume concentration of 75% was used as a mobile phase for elution, and an elution portion containing BEL was collected. The methanol solution was recovered, and a resulting product was dried at about 80° C. in a DHG-9140A electric constant temperature blast drying oven to obtain 48 g of BEL. According to LC3100 HPLC analysis, the BEL had a content of 98.6% by weight.

### Example 2

### 1. A Method for Preparing a BEL-Containing Extract Included the Following Steps:

(1) Extraction: 10 kg of dried whole herb of Swertia mussotii was chopped with a QJY-200 straight-cutting herb cutter, and added with 200 L of 75% ethanol solution at a solid-to-liquid ratio of 1 g: 20 mL. An obtained mixture was subjected to heating reflux extraction at 90° C. in a TQG-0.3-01 extraction tank for 2 times, each time for 2 h. Obtained extracts were combined and subjected to vacuum concentration in an SJN-150-00 double-effect concentrator to recover ethanol. The combined extracts were further concentrated in an R-1020 rotary evaporator to obtain 4.3 kg of a crude extract (with water content of 62%).
(2) Hot-melt and acid hydrolysis: the crude extract in step (1) was dissolved in 50 L of hot water at not less than 90° C. in a F50H2 glass reactor, added with 500 mL of concentrated hydrochloric acid and stirred evenly, and an obtained mixture was subjected to hydrolysis at 90° C. for 0.5 h to obtain an acid hydrolyzate.
(3)Alkali neutralization and salting out precipitation: the acid hydrolyzate in step (2) was neutralized with sodium hydroxide solution to a pH value of 7, then added with 3,000 g of sodium chloride and dissolved by stirring, and an obtained mixture was allowed to stand for 2 h to obtain a precipitate-containing solution.
(4) Centrifugation: the precipitate-containing solution in step (3) was centrifuged using a GQ75 high-speed tubular separator to remove the solution, and then washed with clean water 2 to 3 times to obtain a dark brown precipitate.
(5) Hot-melt extraction and recrystallization: the dark brown precipitate in step (4) was dissolved by heating with a small amount of ethanol solution at a volume concentration of 95.0% or pure methanol, cooled, filtered to obtain a filtrate, the filtrate was added with a half volume of water, stirred evenly, then allowed to stand for not less than 24 h, and a yellow powder was precipitated. The yellow powder was filtered and dried in a DHG-9140A electric constant temperature blast drying oven at about 80° C. to obtain 88 g of the BEL-containing extract. According to LC3100 HPLC analysis, the BEL-containing extract had a BEL content of 45.6% by weight.

### 2. Preparative Liquid Chromatography Purification of the BEL

The BEL-containing extract obtained in step 1 was fully dissolved in methanol with a volume concentration of 85% and then separated and purified using a DAC-HB100 preparative liquid chromatograph. Methanol with a volume concentration of 80% was used as a mobile phase for elution, and an elution portion containing BEL was collected. The methanol solution was recovered, and a resulting product was dried at about 80° C. in a DHG-9140A electric constant temperature blast drying oven to obtain 30 g of BEL. According to LC3100 HPLC analysis, the BEL had a content of 99.0% by weight.

### Example 3

### 1. A Method for Preparing a BEL-Containing Extract Included the Following Steps:

(1) Extraction: 10 kg of dried whole herb of Swertia chirayita was chopped with a QJY-200 straight-cutting herb cutter, and added with 250 L of 65% ethanol solution at a solid-to-liquid ratio of 1 g: 25 mL. An obtained mixture was subjected to heating reflux extraction at 90° C. in a TQG-0.3-01 extraction tank 1 time for 2 h. Obtained extracts were combined and subjected to vacuum concentration in an SJN-150-00 double-effect concentrator to recover ethanol. The combined extracts were further concentrated in an R-1020 rotary evaporator to obtain 4.0 kg of a crude extract (with water content of 68%).
(2) Hot-melt and acid hydrolysis: the crude extract in step (1) was dissolved in 50 L of hot water at not less than 90° C. in a F50H2 glass reactor, added with 500 mL of concentrated hydrochloric acid and stirred evenly, and an obtained mixture was subjected to hydrolysis at 90° C. for 1 h to obtain an acid hydrolyzate.
(3)Alkali neutralization and salting out precipitation: the acid hydrolyzate in step (2) was neutralized with sodium hydroxide solution to a pH value of 7, then added with 3,000 g of sodium chloride and dissolved by stirring, and an obtained mixture was allowed to stand for 2 h to obtain a precipitate-containing solution.
(4) Centrifugation: the precipitate-containing solution in step (3) was centrifuged using a GQ75 high-speed tubular separator to remove the solution, and then washed with clean water 2 to 3 times to obtain a dark brown precipitate.
(5) Hot-melt extraction and recrystallization: the dark brown precipitate in step (4) was dissolved by heating with a small amount of ethanol solution at a volume concentration of 95.0% or pure methanol, cooled, filtered to obtain a filtrate, the filtrate was added with a half volume of water, stirred evenly, then allowed to stand for not less than 24 h, and a yellow powder was precipitated. The yellow powder was filtered and dried in a DHG-9140A electric constant temperature blast drying oven at about 80° C. to obtain 70 g of the BEL-containing extract. According to LC3100 HPLC analysis, the BEL-containing extract had a BEL content of 56.0% by weight.

### 2. Preparative Liquid Chromatography Purification of the BEL

The BEL-containing extract obtained in step 1 was fully dissolved in methanol with a volume concentration of 85% and then separated and purified using a DAC-HB100 preparative liquid chromatograph. Methanol with a volume concentration of 85% was used as a mobile phase for elution, and an elution portion containing BEL was collected. The methanol solution was recovered, and a resulting product was dried at about 80° C. in a DHG-9140A electric constant temperature blast drying oven to obtain 32 g of BEL. According to LC3100 HPLC analysis, the BEL had a content of 99.2% by weight.

### Example 4

### 1. A Method for Preparing a BEL-Containing Extract Included the Following Steps:

(1) Extraction: 10 kg of dried whole herb of Lomatogonium carinthiacum was chopped with a QJY-200 straight-cutting herb cutter, and added with 250 L of 75% ethanol solution at a solid-to-liquid ratio of 1 g: 25 mL. An obtained mixture was subjected to heating reflux extraction at 90° C. in a TQG-0.3-01 extraction tank for 2 times, each time for 2 h. Obtained extracts were combined and subjected to vacuum concentration in an SJN-150-00 double-effect concentrator to recover ethanol. The combined extracts were further concentrated in an R-1020 rotary evaporator to obtain 6.0 kg of a crude extract (with water content of 63%).
(2) Hot-melt and acid hydrolysis: the crude extract in step (1) was dissolved in 50 L of hot water at not less than 90° C. in a F50H2 glass reactor, added with 500 mL of concentrated hydrochloric acid and stirred evenly, and an obtained mixture was subjected to hydrolysis at 90° C. for 1 h to obtain an acid hydrolyzate.
(3)Alkali neutralization and salting out precipitation: the acid hydrolyzate in step (2) was neutralized with sodium hydroxide solution to a pH value of 7, then added with 3,000 g of sodium chloride and dissolved by stirring, and an obtained mixture was allowed to stand for 2 h to obtain a precipitate-containing solution.
(4) Centrifugation: the precipitate-containing solution in step (3) was centrifuged using a GQ75 high-speed tubular separator to remove the solution, and then washed with clean water 2 to 3 times to obtain a dark brown precipitate.
(5) Hot-melt extraction and recrystallization: the dark brown precipitate in step (4) was dissolved by heating with a small amount of pure methanol, cooled, filtered to obtain a filtrate, the filtrate was added with an equal volume of water, stirred evenly, then allowed to stand for not less than 24 h, and a yellow powder was precipitated. The yellow powder was filtered and dried in a DHG-9140A electric constant temperature blast drying oven at about 80° C. to obtain 105 g of the BEL-containing extract. According to LC3100 HPLC analysis, the BEL-containing extract had a BEL content of 46.4% by weight.

### 2. Preparative Liquid Chromatography Purification of the BEL

The BEL-containing extract obtained in step 1 was fully dissolved in methanol with a volume concentration of 85% and then separated and purified using a DAC-HB100 preparative liquid chromatograph. Methanol with a volume concentration of 82% was used as a mobile phase for elution, and an elution portion containing BEL was collected. The methanol solution was recovered, and a resulting product was dried at about 80° C. in a DHG-9140A electric constant temperature blast drying oven to obtain 38 g of BEL. According to LC3100 HPLC analysis, the BEL had a content of 97.6% by weight.

### Example 5

### 1. A Method for Preparing a BEL-Containing Extract Included the Following Steps:

(1) Extraction: 10 kg of dried whole herb of Swertia franchetiana was chopped with a QJY-200 straight-cutting herb cutter, and added with 300 L of 70% ethanol solution at a solid-to-liquid ratio of 1 g: 30 mL. An obtained mixture was subjected to heating reflux extraction at 90° C. in a TQG-0.3-01 extraction tank for 3 times, each time for 2 h. Obtained extracts were combined and subjected to vacuum concentration in an SJN-150-00 double-effect concentrator to recover ethanol. The combined extracts were further concentrated in an R-1020 rotary evaporator to obtain 6.8 kg of a crude extract (with water content of 65%).
(2) Hot-melt and acid hydrolysis: the crude extract in step (1) was dissolved in 50 L of hot water at not less than 90° C. in a F50H2 glass reactor, added with 500 mL of concentrated hydrochloric acid and stirred evenly, and an obtained mixture was subjected to hydrolysis at 90° C. for 1 h to obtain an acid hydrolyzate.
(3)Alkali neutralization and salting out precipitation: the acid hydrolyzate in step (2) was neutralized with sodium hydroxide solution to a pH value of 7, then added with 3,000 g of sodium chloride and dissolved by stirring, and an obtained mixture was allowed to stand for 2 h to obtain a precipitate-containing solution.
(4) Centrifugation: the precipitate-containing solution in step (3) was centrifuged using a GQ75 high-speed tubular separator to remove the solution, and then washed with clean water 2 to 3 times to obtain a dark brown precipitate.
(5) Hot-melt extraction and recrystallization: the dark brown precipitate in step (4) was dissolved by heating with a small amount of pure methanol, cooled, filtered to obtain a filtrate, the filtrate was added with a half volume of water, stirred evenly, then allowed to stand for not less than 24 h, and a yellow powder was precipitated. The yellow powder was filtered and dried in a DHG-9140A electric constant temperature blast drying oven at about 80° C. to obtain 22 g of the BEL-containing extract. According to LC3100 HPLC analysis, the BEL-containing extract had a BEL content of 95.0% by weight.

### 2. Preparative Liquid Chromatography Purification of the BEL

The BEL-containing extract obtained in step 1 was fully dissolved in methanol with a volume concentration of 85% and then separated and purified using a DAC-HB100 preparative liquid chromatograph. Methanol with a volume concentration of 88% was used as a mobile phase for elution, and an elution portion containing BEL was collected. The methanol solution was recovered, and a resulting product was dried at about 80° C. in a DHG-9140A electric constant temperature blast drying oven to obtain 19 g of BEL. According to LC3100 HPLC analysis, the BEL had a content of 99.9% by weight.

### Example 6

### 1. A Method for Preparing a BEL-Containing Extract Included the Following Steps:

(1) Extraction: 10 kg of dried whole herb of Halenia elliptica was chopped with a QJY-200 straight-cutting herb cutter, and added with 250 L of 65% ethanol solution at a solid-to-liquid ratio of 1 g: 25 mL. An obtained mixture was subjected to heating reflux extraction at 90° C. in a TQG-0.3-01 extraction tank 1 time for 2 h. Obtained extracts were combined and subjected to vacuum concentration in an SJN-150-00 double-effect concentrator to recover ethanol. The combined extracts were further concentrated in an R-1020 rotary evaporator to obtain 4.1 kg of a crude extract (with water content of 65%).
(2) Hot-melt and acid hydrolysis: the crude extract in step (1) was dissolved in 50 L of hot water at not less than 90° C. in a F50H2 glass reactor, added with 500 mL of concentrated hydrochloric acid and stirred evenly, and an obtained mixture was subjected to hydrolysis at 90° C. for 1 h to obtain an acid hydrolyzate.
(3)Alkali neutralization and salting out precipitation: the acid hydrolyzate in step (2) was neutralized with sodium hydroxide solution to a pH value of 7, then added with 3,000 g of sodium chloride and dissolved by stirring, and an obtained mixture was allowed to stand for 2 h to obtain a precipitate-containing solution.
(4) Centrifugation: the precipitate-containing solution in step (3) was centrifuged using a GQ75 high-speed tubular separator to remove the solution, and then washed with clean water 2 to 3 times to obtain a dark brown precipitate.
(5) Hot-melt extraction and recrystallization: the dark brown precipitate in step (4) was dissolved by heating with a small amount of ethanol solution at a volume concentration of 95.0% or pure methanol, cooled, filtered to obtain a filtrate, the filtrate was added with a half volume of water, stirred evenly, then allowed to stand for not less than 24 h, and a yellow powder was precipitated. The yellow powder was filtered and dried in a DHG-9140A electric constant temperature blast drying oven at about 80° C. to obtain 52 g of the BEL-containing extract. According to LC3100 HPLC analysis, the BEL-containing extract had a BEL content of 1.0% by weight.
   2. Preparative liquid chromatography purification of the BEL

The BEL-containing extract obtained in step 1 was fully dissolved in methanol with a volume concentration of 85% and then separated and purified using a DAC-HB100 preparative liquid chromatograph. Methanol with a volume concentration of 80% was used as a mobile phase for elution, and an elution portion containing BEL was collected. The methanol solution was recovered, and a resulting product was dried at about 80° C. in a DHG-9140A electric constant temperature blast drying oven to obtain 0.37 g of BEL. According to LC3100 HPLC analysis, the BEL had a content of 95.0% by weight.

### Test Example 1

### In Vitro Antidepressant Activity Test of BEL

1. Construction of HT22 cell model

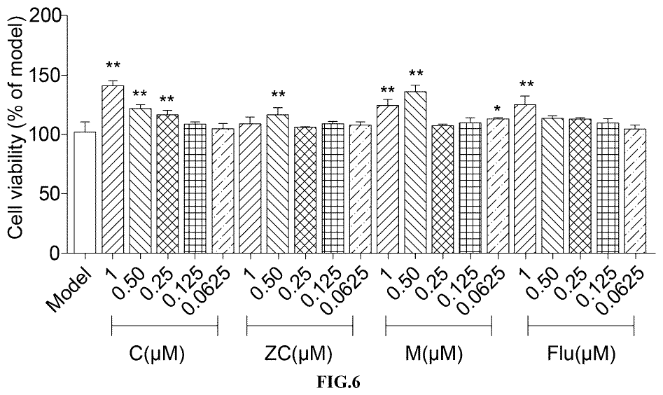

HT22 cells were induced with 350 μM, 300 μM, 250 μM, 200 μM, and 150 M of CORT to explore an influence of different concentrations of CORT on HT22 cell proliferation. The results were shown in FIG. 6. According to FIG. 6, a CORT induction concentration was determined to be 250 M based on the condition that the cell activity in the induced group was greater than 50% and less than 75%. Subsequently, the HT22 cell model induced by 250 M CORT was used for later experiments.
2. HT22 cells were randomly divided into a model group (model), a bel group (c), and a fluoxetine hydrochloride group (flu); where
model group: the inducer (CORT) was applied to HT22 cells for 24 h, where CORT had a final concentration of 250 M;
BEL group (C): the inducer (CORT) and the BEL obtained in Example 5 were co-acted on HT22 cells for 24 h, where CORT had a final concentration of 250 μM, and BEL had a final concentration of 1 μM, 0.5 μM, 0.25 μM, 0.125 μM, or 0.0625 M;
fluoxetine hydrochloride group (Flu): the inducer (CORT) and fluoxetine hydrochloride were co-acted on HT22 cells for 24 h, where CORT had a final concentration of 250 μM, and fluoxetine hydrochloride had a final concentration of 1 μM, 0.5 μM, 0.25 μM, 0.125 μM, or 0.0625 μM.

After the treatment of each treatment group, the HT22 cell viability of each treatment group was counted. The results were shown in Table 1 and FIG. 6.

TABLE 1
HT22 cell viability induced by CORT in different treatment groups (x̅ ± SD)

| | Concentration/(μM) | | | | |
|---|---|---|---|---|---|
| Treatment group | 0.0625 | 0.125 | 0.25 | 0.5 | 1 |
| Model | 100.00 ± 9.31 | | | | |
| C | 103.02 ± 5.03 | 106.88 ± 2.64 | 114.84 ± 3.93** | 120.43 ± 3.97** | 139.30 ± 5.34** |
| Flu | 102.88 ± 4.06 | 108.23 ± 3.95 | 111.20 ± 1.64 | 111.84 ± 2.77 | 123.38 ± 8.39** |

NOTE:
compared with the model group,
*P < 0.05,
**P < 0.01.

As shown in Table 1 and FIG. 6, BEL could significantly improve the viability of HT22 cells induced by CORT and had the best effect.

### Test Example 2

### Acute Toxicity Study of BEL

After one week of adaptive feeding, the experimental mice were randomly divided into a blank group (Control), a solvent control group (Solvent), and a treatment group (Treated), with 5 mice in each group. The blank group was not treated, the solvent control group was gavaged with normal saline (0.9%) containing 1% DMSO+2% Tween 80, and the treatment group was gavaged with 214 mg/kg of the solution of BEL obtained in Example 5 according to the maximum drug dissolution dose. The mice were starved for 24 h before the experiment, and then the drug was treated 3 times within 24 h. After the drug treatment, the mice were closely observed for 2 h, and then observed continuously for 14 d. The results showed that the mice did not show any abnormalities within 2 h after the first and third treatments. 20 min after the second treatment, the mice showed slight tremors, which disappeared after 2 min, and then the mice returned to normal. The mice did not show any abnormalities during the subsequent 14-d observation period.

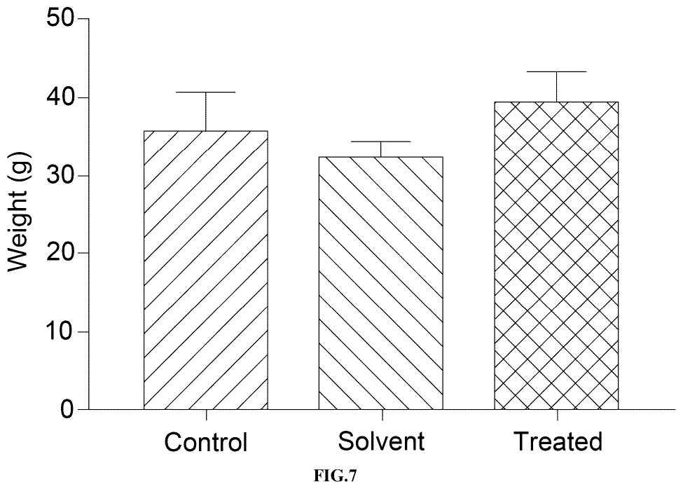

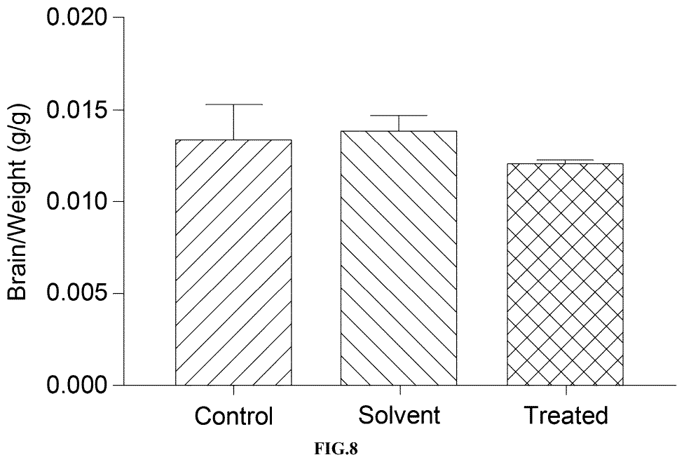

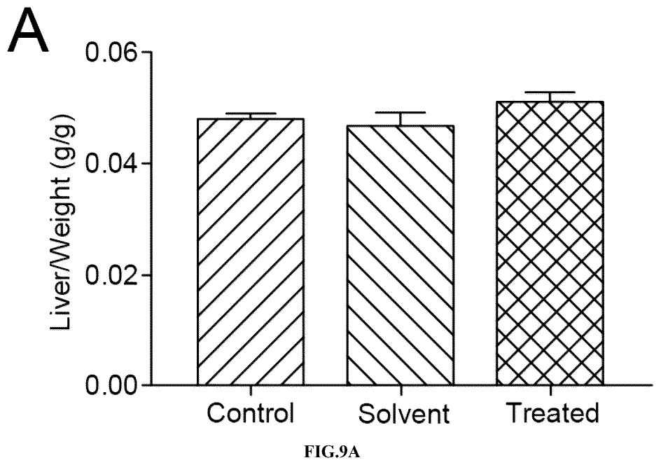

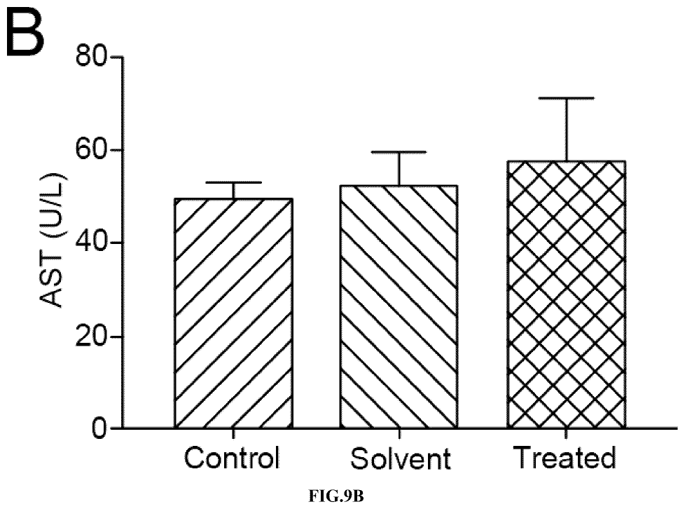

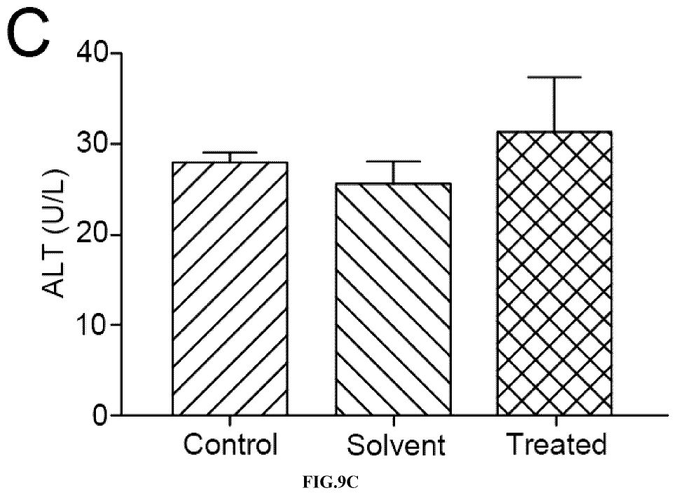

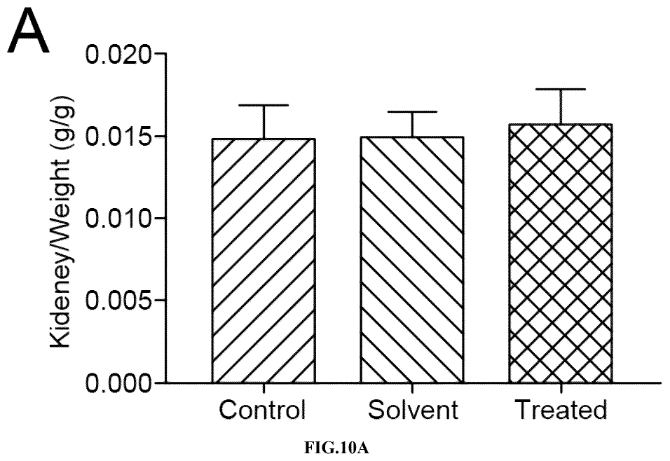

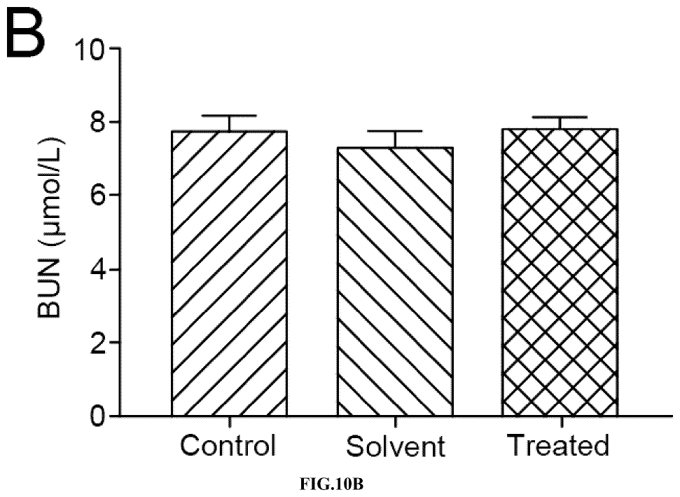

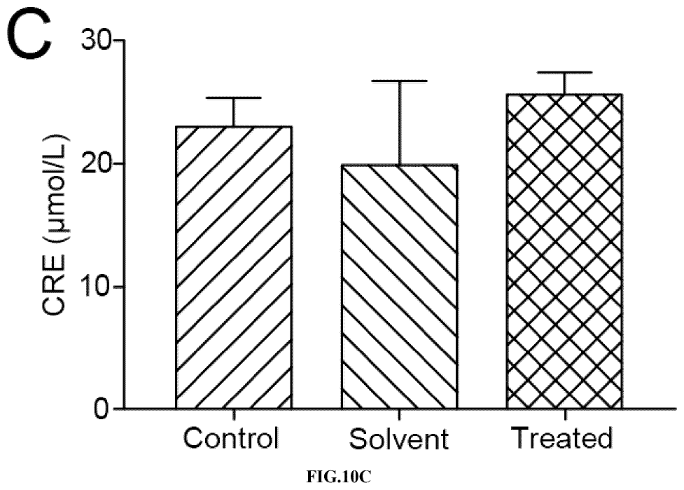

After the treatments, the body weight (weight), brain index (Brain/weight), leptosomatic index (Liver/weight), serum AST level (U/L), serum ALT level (U/L), kidney index (Kidney/weight), serum BUN level (μmol/L), and serum CRE level (μmol/L) of the mice were recorded every day. The results were shown in Table 2 and FIG. 7 to FIG. 10C.

TABLE 2
Test results of various test indicators of mice in
each treatment group (x̅ ± SD)

| Group | Control | Solvent | Treated |
|---|---|---|---|
| Weight (g) | 35.30 ± 5.30 | 32.04 ± 2.28 | 39.18 ± 3.94 |
| Brain/weight (g/g) | 1.32 ± 0.20 | 1.37 ± 0.09 | 1.10 ± 0.17 |
| Liver/weight (g/g) | 4.76 ± 0.13 | 4.65 ± 0.24 | 5.05 ± 0.22 |
| AST (U/L) | 47.56 ± 2.09 | 49.44 ± 7.26 | 48.73 ± 4.15 |
| ALT (U/L) | 25.33 ± 2.65 | 22.37 ± 3.09 | 26.85 ± 6.22 |
| Kidney/weight (g/g) | 1.47 ± 0.20 | 1.49 ± 0.16 | 1.56 ± 0.22 |
| BUN (μmol/L) | 7.68 ± 0.45 | 7.25 ± 0.47 | 8.06 ± 0.57 |
| CRE (μmol/L) | 26.42 ± 1.72 | 18.25 ± 6.21 | 23.20 ± 5.69 |

According to Table 2 and FIG. 7 to FIG. 10C: Under the action of BEL (treatment group), the changes in mouse body weight and brain index compared with those in the blank group and solvent control group were not statistically significant, indicating that BEL had no effect on the weight and brain of mice. Under the action of BEL (treatment group), the changes in mouse hepatosomatic index and serum AST and ALT levels compared with those in the blank group and solvent control group were not statistically significant, indicating that BEL had no effect on mouse liver. Under the action of BEL (treatment group), the changes in mouse kidney index and serum BUN and CRE levels compared with those in the blank group and solvent control group were not statistically significant, indicating that BEL had no effect on mouse kidneys. This proved that BEL had no toxic side effects on mice. This was mainly manifested in that BEL showed no significant effect on the weight, brain, liver, and kidneys of mice.

### Test Example 3

### In Vivo Antidepressant Effect Study of BEL

The in vivo antidepressant effect study was conducted according to the experimental procedure shown in FIG. 11, specifically:

After one week of adaptive feeding of experimental mice, 10 mice were randomly selected from 150 mice as a normal group, and the mice in the normal group were not treated in any way. The remaining mice were used as a modeling group, and the mice in the modeling group were induced with CORT for 21 d, with a CORT dose of 20 mg/kg and a dosing volume of 0.1 mL/20 mg. The dosing volume was changed once a week according to the weight of the mice, and the mice were subcutaneously administered in the groin of the hind limb between 9:00 and 11:00 am every day. During the modeling, the weight, diet, and water intake of the mice were recorded, and the relevant data were counted once a week until the modeling was completed. During the modeling, the mice were subjected to behavioral analysis. 4 behavioral test designs were designed: FST, TST, SPT, and NSFT. Behavioral tests were conducted before the model was induced, and behavioral experiments were conducted 1 time one week after the stimulation began; the body weight was measured once a week, and the drug was administered for a total of 3 weeks. There were 5 mice/cage in the normal group and 5 mice/cage in the model group.

After the model induction was completed, 60 mice with successful modeling were screened by behavioral indicators and divided into: a model group, a positive drug imipramine hydrochloride (Imi) group (20 mg/kg/d, gavage), a positive drug fluoxetine hydrochloride (Flu) group (20 mg/kg/d, gavage), a BEL high-dose (CH) group (40 mg/kg/d, gavage), a BEL medium-dose (CM) group (20 mg/kg/d, gavage), and a BEL low-dose (CL) group (10 mg/kg/d, gavage); where the BEL used was the BEL obtained in Example 5. The normal group received no treatment, the model group was gavaged with normal saline (0.9%) containing 1% DMSO+2% Tween 80, the positive drug Imi and Flu groups were gavaged with Imi and Flu solutions, respectively, the BEL high-dose group was gavaged with 40 mg/kg/d, the BEL medium-dose group was gavaged with 20 mg/kg/d, and the BEL low-dose group was gavaged with 10 mg/kg/d. Except for the mice in normal group, the other mice were gavaged at 9:00 to 11:00 every morning, and CORT was injected subcutaneously 2 h after the end of gavage for a total of 21 d.
(1) Observation of the status of mice in each group during drug administration

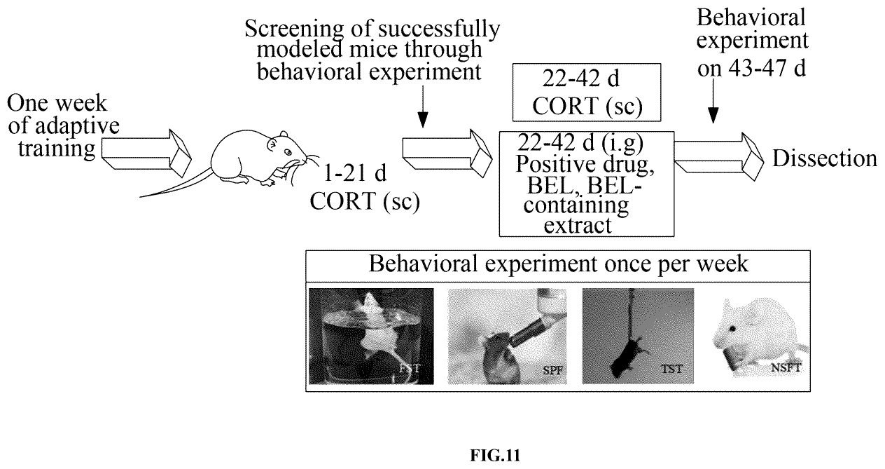

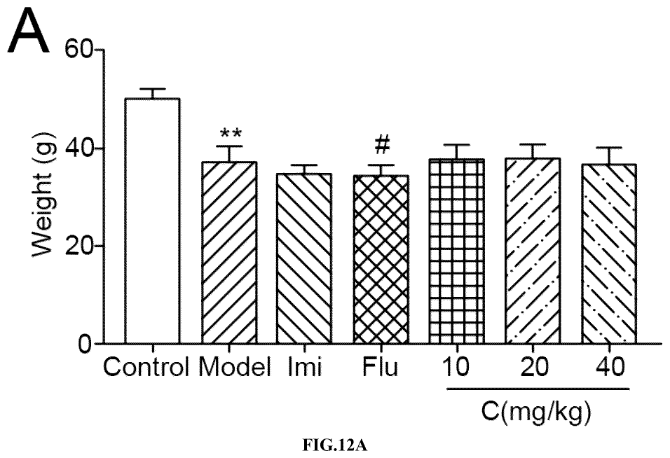

During the administration period, the hair and urine color of the mice remained normal and no diarrhea was observed.
(2) During the administration, body weight, diet, and water intake were recorded weekly, and the administration volume was changed once a week. Behavioral experiments were conducted once a week, and the serum 5-HT and CORT levels of mice were recorded. The results were shown in Tables 3 to 5 and FIG. 12A to FIG. 14B.

TABLE 3
Body weight, diet, and water intake of mice in each
treatment group (x̅ ± SD)

| Group | Body weight (g) | Diet (g/d) | Water intake (g/d) |
|---|---|---|---|
| Control | 49.84 ± 2.12 | 5.95 ± 0.15 | 6.00 ± 0.29 |
| Model | 34.29 ± 2.65# | 4.65 ± 0.45 | 5.66 ± 0.27 |
| Imi | 34.41 ± 1.80 | 4.38 ± 0.45 | 4.92 ± 0.40 |
| Flu | 27.03 ± 1.74* | 3.90 ± 0.39 | 4.00 ± 0.97 |
| CL | 37.19 ± 3.35 | 4.02 ± 0.03 | 4.72 ± 0.42 |
| CM | 37.33 ± 3.30 | 4.48 ± 0.45 | 4.87 ± 0.99 |
| CH | 36.31 ± 3.72 | 5.14 ± 1.00 | 6.82 ± 0.45 |

NOTE:
compared with the control group,
#P < 0.01, compared with the model group,
*P < 0.05.

According to Table 3 and FIGS. 12(A-C), compared with the normal group, the model group had a significant decrease in body weight (P<0.01), and there was no statistically significant change in diet and water intake. Compared with the model group, BEL at 10, 20, 40 mg/kg and the positive control imipramine hydrochloride group had no significant effect on the body weight, diet, and water intake of mice. The body weight of mice in the positive control fluoxetine hydrochloride group was lower than that in the model group, and there was no significant effect on diet and water intake. The reason was that the side effects of fluoxetine hydrochloride in the treatment of depression caused the weight loss of the mice. BEL had no effect on the body weight of depressed mice.

TABLE 4
Results of behavioral test of mice in each treatment group (x̅ ± SD)

| Group | FST (s) | TST (s) | SPT (%) | NSFT (s) |
|---|---|---|---|---|
| Control | 176.67 ± 43.20 | 95.00 ± 14.59 | 17.09 ± 1.07 | 65.11 ± 31.14 |
| Model | 207.50 ± 18.64# | 156.67 ± 47.19# | 10.37 ± 1.58# | 103.00 ± 25.82# |
| Imi | 163.00 ± 38.09* | 76.43 ± 46.52* | 8.32 ± 0.12 | 65.11 ± 32.90* |
| Flu | 178.50 ± 36.52 | 71.43 ± 40.80* | 13.28 ± 0.56* | 84.50 ± 32.69 |
| CL | 145.00 ± 36.40* | 27.50 ± 16.05* | 13.72 ± 0.30* | 42.75 ± 31.56* |
| CM | 127.14 ± 36.38* | 47.22 ± 35.19* | 12.41 ± 3.32 | 57.14 ± 35.77* |
| CH | 193.33 ± 25.98 | 98.75 ± 66.35 | 8.75 ± 0.18 | 38.78 ± 22.73* |

NOTE:
compared with the control group,
#P < 0.05, compared with the model group,
*P < 0.05.

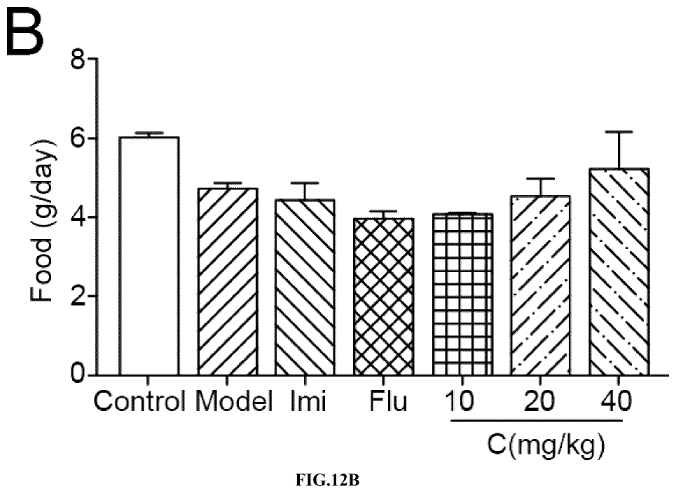

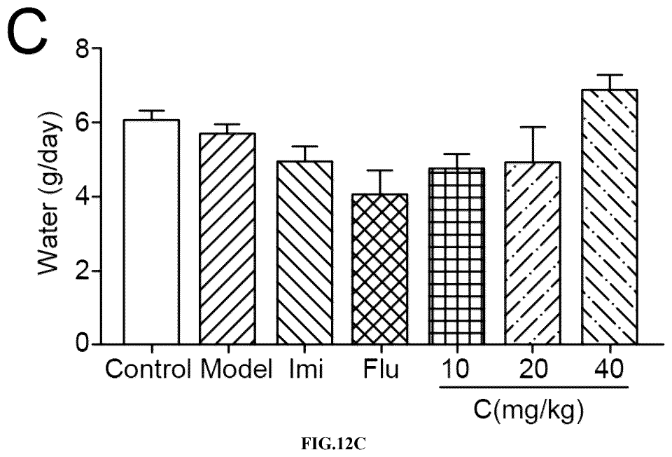

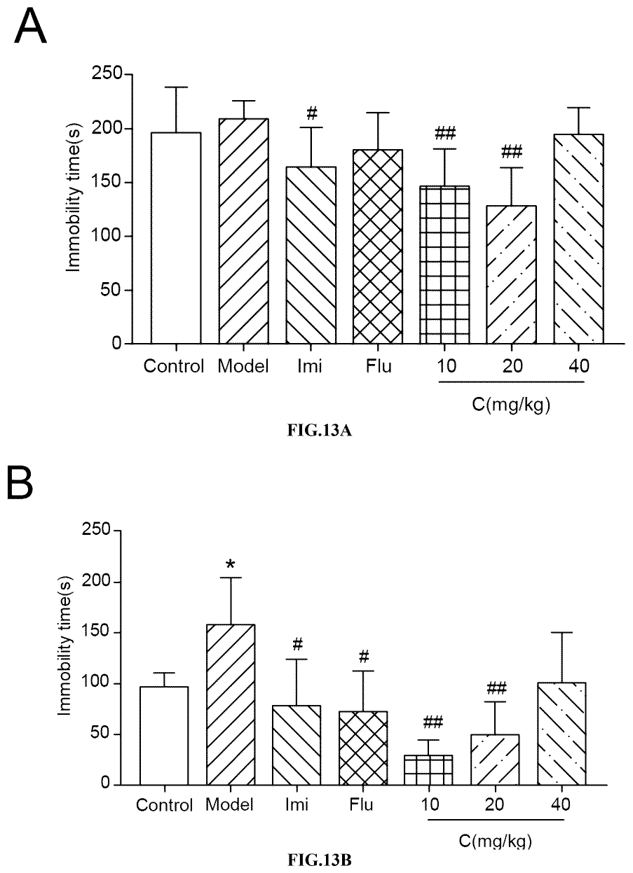

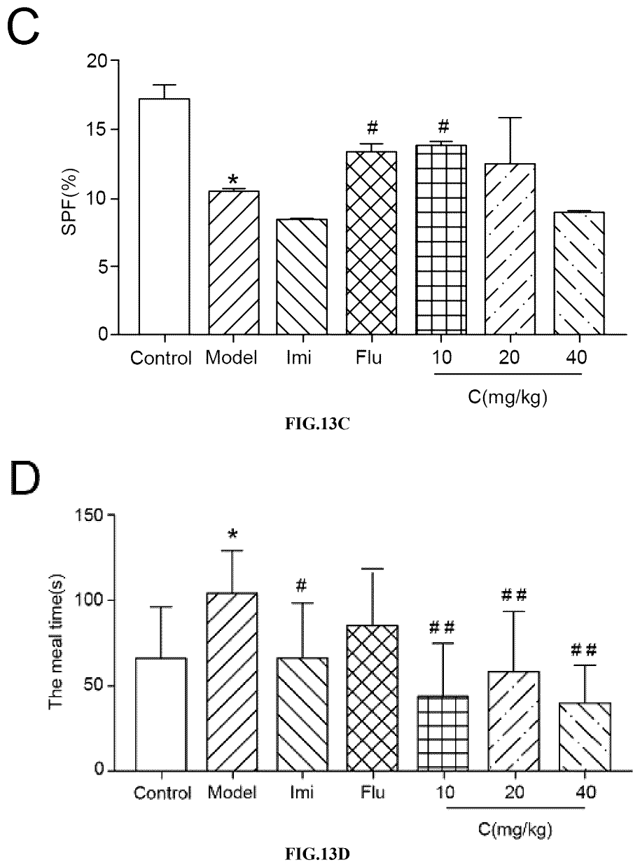

As shown in Table 4 and FIGS. 13(A-D), compared with the normal group, the immobility time in FST (Forced swimming test) and TST (Tail suspension test) of the model group was significantly increased, the preference for sugar water in SPT (sucrose preference test) was reduced, and the eating time of mice in NSFT (Novelty-Suppressed Feeding Test) was significantly increased, showing obvious depressive-like behavior. Compared with the model group, BEL at 10 and 20 mg/kg could significantly reduce the immobility time of mice in FST and TST, BEL at 10 mg/kg could significantly increase the preference for sugar water of mice, and BEL at 10, 20, and 40 mg/kg could significantly reduce the time for mice to start eating. The results showed that BEL could significantly improve the depressive-like behavior of mice.

TABLE 5
Serum 5-HT and CORT levels of mice in each treatment
group (x̅ ± SD)

| Group | 5-HT (ng/mL) | CORT (ng/mL) |
|---|---|---|
| Control | 157.64 ± 33.87 | 126.87 ± 10.41 |
| Model | 102.47 ± 14.18# | 186.33 ± 13.58## |
| Imi | 130.24 ± 13.65 | 143.19 ± 12.30** |
| Flu | 142.08 ± 11.12* | 152.77 ± 11.25** |
| CL | 134.29 ± 12.34 | 107.08 ± 8.16** |
| CM | 216.11 ± 3.34** | 111.82 ± 8.27** |
| CH | 133.42 ± 30.76 | 93.63 ± 11.48** |

NOTE:
compared with the control group,
#P < 0.05,
##P < 0.01; compared with the model group,
*P < 0.05,
**P < 0.01.

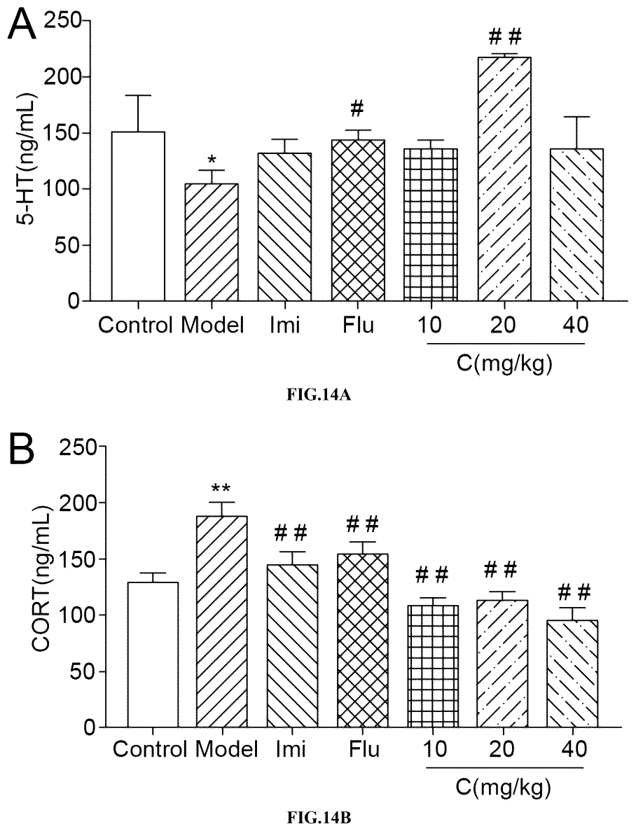

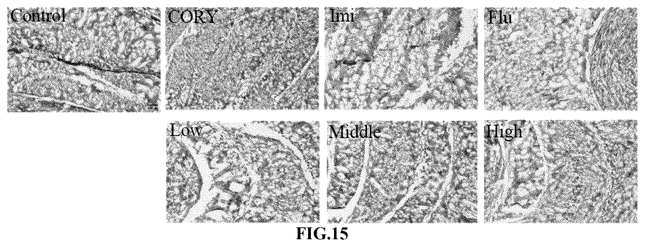

As shown in Table 5 and FIGS. 14(A-B), compared with the normal group, the serum 5-HT level in the model group was significantly reduced, while the CORT level was significantly increased. Compared with the model group, BEL at 20 mg/kg could significantly increase the serum 5-HT level, and BEL at 10, 20, and 40 mg/kg could significantly reduce the serum CORT level of mice. This indicated that BEL could achieve antidepressant effects by regulating the synthesis of 5-HT and CORT. [0170](2) After 21 d of drug administration, the hippocampus of mice in each treatment group was stained with H & E, and the proliferation of neurons in the hippocampus of mice was detected by immunofluorescence histochemistry. The results were shown in FIG. 15 to FIG. 17.

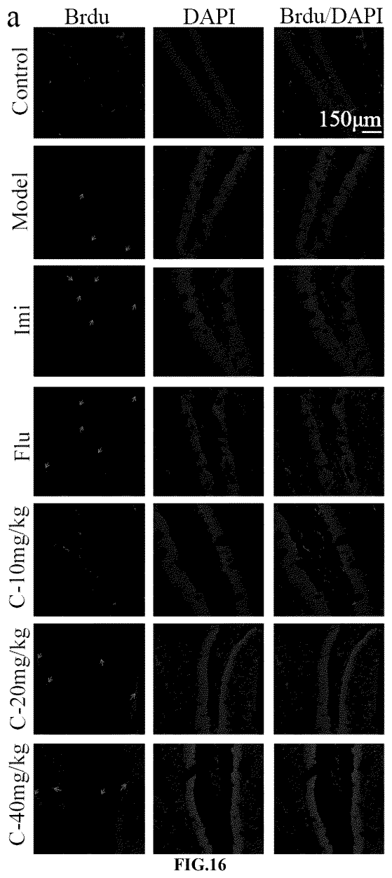

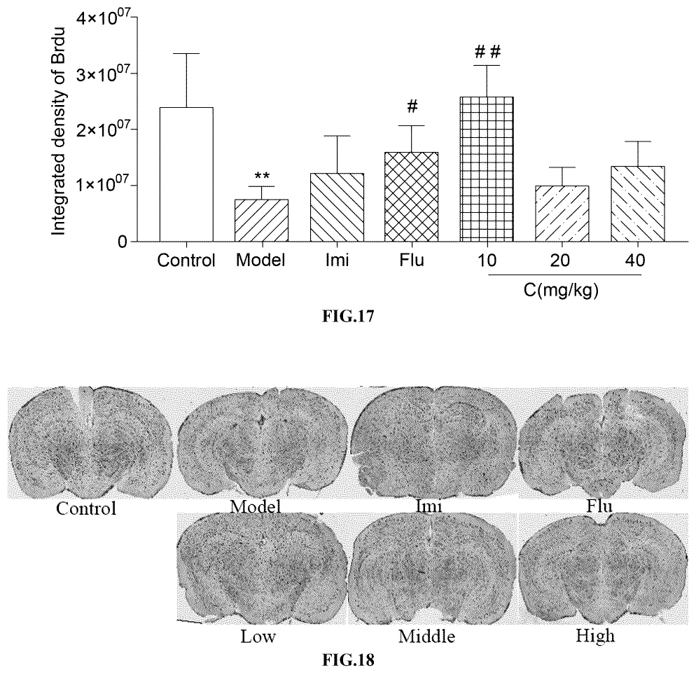

As shown in FIG. 17, the hippocampus structure of the normal group mice was normal, the cells were evenly distributed and then neatly and densely arranged, and the cell appearance was regular. Compared with the normal group, the hippocampus structure of the model group mice was abnormal, the cells were wrinkled and then irregularly arranged. After administration, the hippocampus structure returned to normal and the cells were arranged regularly. The number of neurons in the model group mice was significantly reduced, indicating that CORT induction could significantly affect the regeneration and maturation of neurons in the mouse hippocampus. The immunohistofluorescence showed that proliferation of neurons in the mouse was inhibited, and BEL could promote the proliferation of neurons in the mouse hippocampus. [0172](3) After 21 d of drug administration, brain tissues of mice in each treatment group were collected for Golgi staining. The results showed that the number of neurons in the hippocampus of mice in the positive model group was reduced and the cell appearance was shorter. BEL at 10 and 20 mg/kg increased the number of neurons and the length of neurons (FIG. 18 and Table 6).

TABLE 6
Increase in dendrite length of neurons in mouse hippocampus
in each treatment group (x̅ ± SD)

| | Neuron dendrite length (μm) | | |
|---|---|---|---|
| Group | Mean | Minimum | Maximum |
| Control | 57.90 ± 34.46 | 9.04 | 216.55 |
| Model | 48.71 ± 32.76 | 6.89 | 186.00 |
| Imi | 37.66 ± 25.07 | 5.95 | 172.10 |
| Flu | 54.65 ± 32.76 | 6.57 | 180.19 |
| CL | 75.66 ± 60.55 # | 5.86 | 1853.00 |
| CM | 140.64 ± 73.11 ## | 6.32 | 2297.92 |
| CH | 51.73 ± 33.73 | 6.04 | 194.06 |

NOTE:
compared with the control group, compared with the model group,
# P < 0.05,
## P < 0.01.

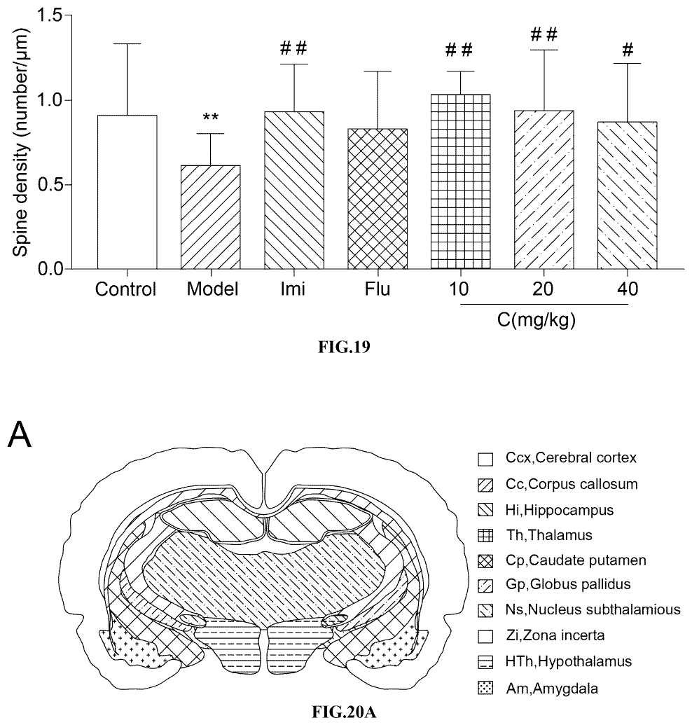

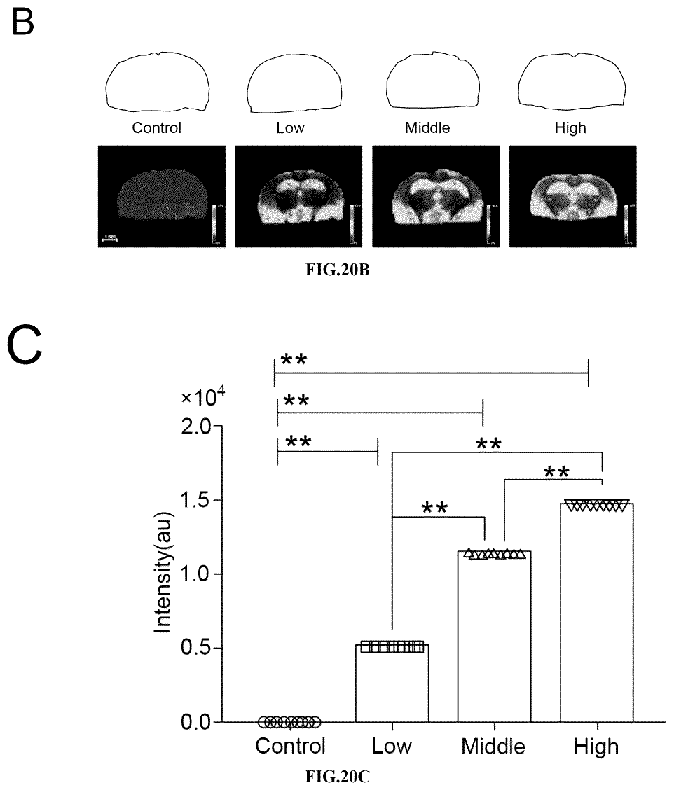

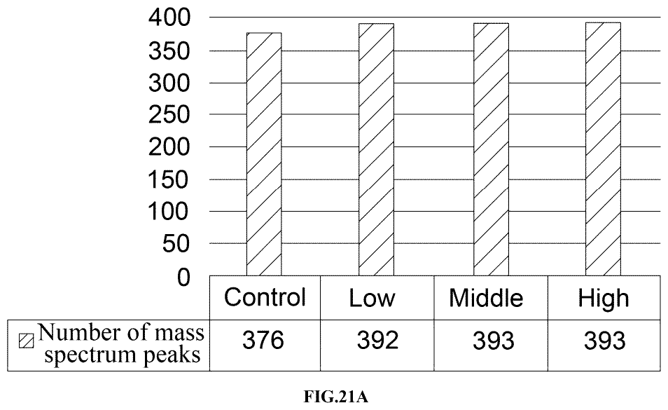

(4) After 21 d of drug administration, the dendritic spine density of neurons in the hippocampus of mice was counted. The results were shown in FIG. 19. Compared with the normal group, the dendritic spine density of neurons in the hippocampus of the model group was significantly reduced, while the 10, 20, and 40 mg/kg BEL treatment groups could significantly increase the dendritic spine density. The effect intensity was as follows: 10 mg/kg BEL treatment group >20 mg/kg BEL treatment group BEL 40 mg/kg BEL treatment group. BEL could significantly increase the dendritic spine density of neurons in the hippocampus of mice.
(5) After 21 d of drug administration, the brains of mice in each treatment group were collected for mass spectrometry imaging. The results showed that no BEL signal was detected in the brain of the normal group, while BEL signals were detected in the 10, 20, and 40 mg/kg BEL treatment groups, indicating that the target compound could act on the brain through the BBB. BEL was mainly distributed in the hypothalamus, amygdala, hippocampus, and cortex of mice. BEL expression abundance in different parts decreased successively, and BEL accumulation in the brain increased significantly with the increase of compound dosage (FIGS. 20(A-C)).

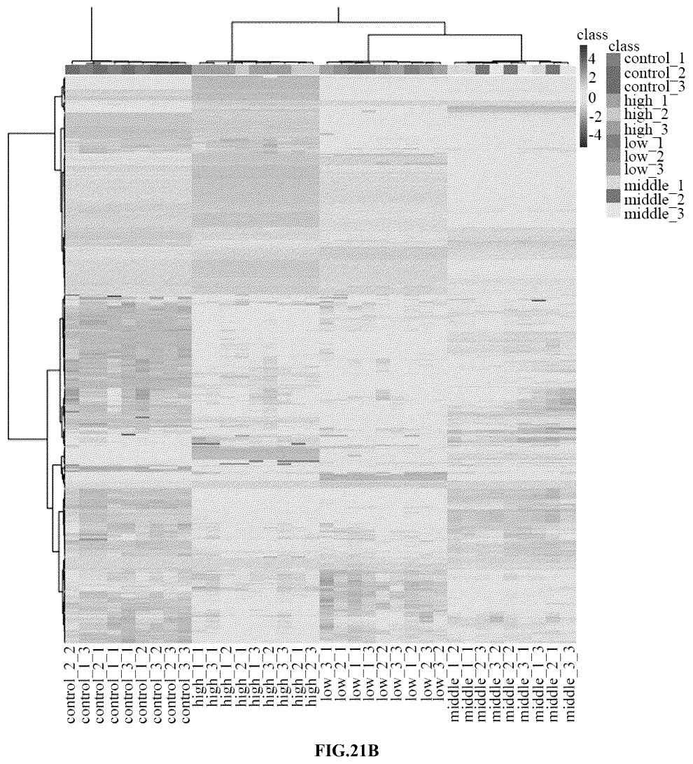

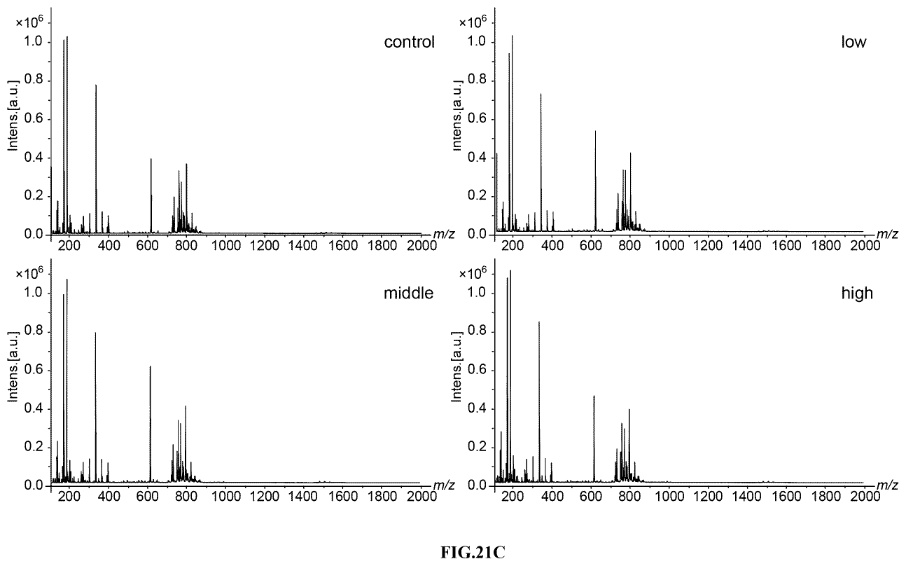

Further detection and analysis of ions at 100 Da to 2,000 Da in the brain showed that there were differences in the number of ions between the normal group and the compound treatment group, but the difference in the number of ions within the compound treatment group was not significant. There were fewer ions at 100 Da to 900 Da with higher expression abundance, while there were fewer ions at 900 Da to 2,000 Da with lower expression abundance. Among these ions, 350 ions were common to all groups, the normal group had 5 unique ions, the low-dose group had 5 unique ions, the medium-dose group had no unique ions, the high-dose group had 9 unique ions, and the high-, medium-, and low-dose groups of the compound had 10 common ions (FIG. 21A, FIG. 21B, and FIG. 21C).

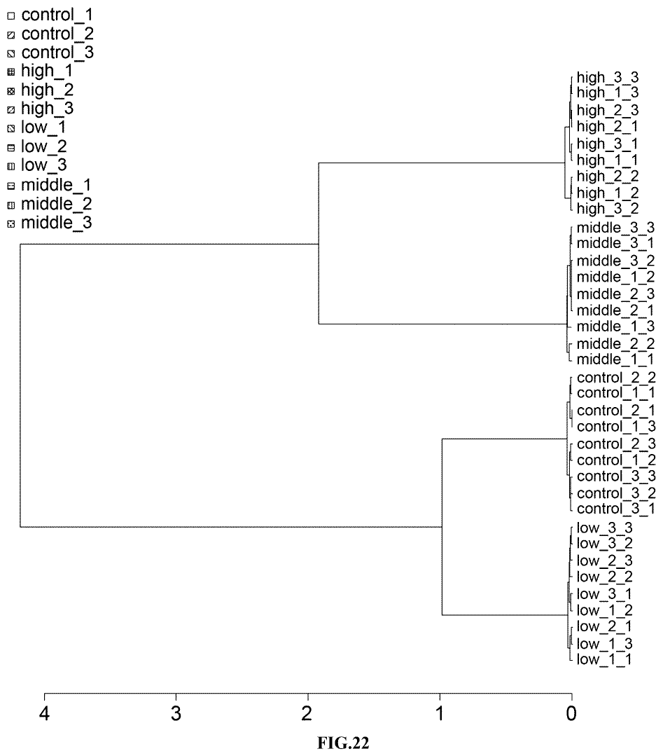

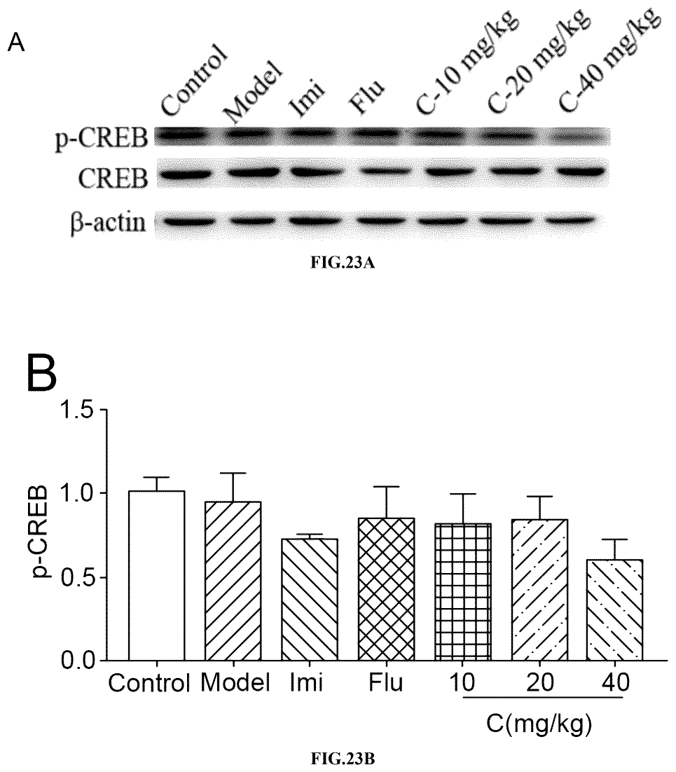

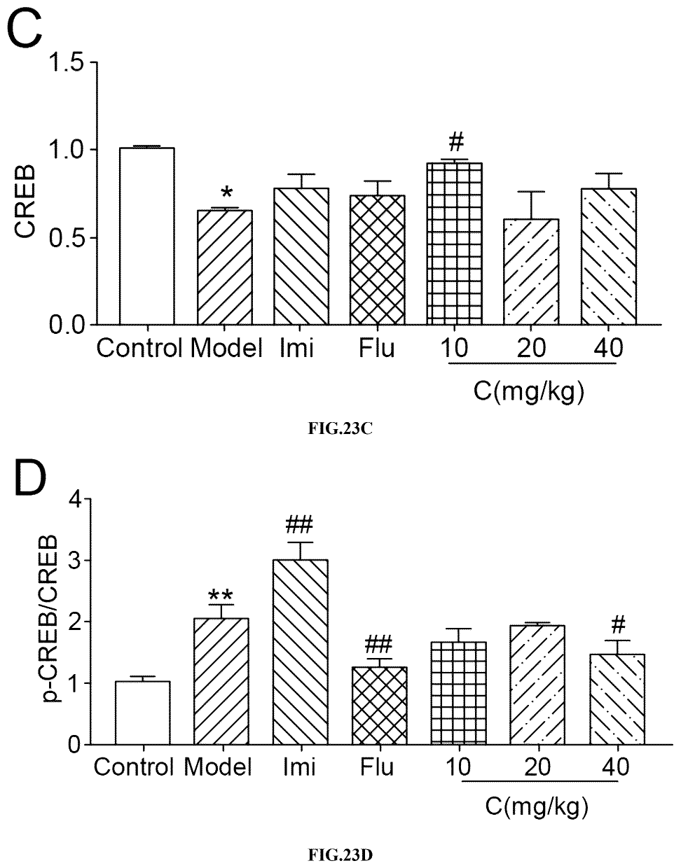

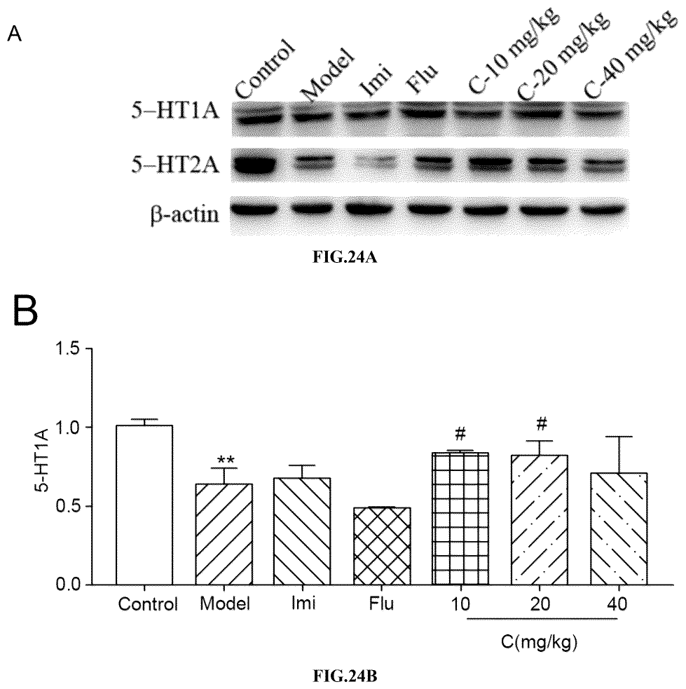

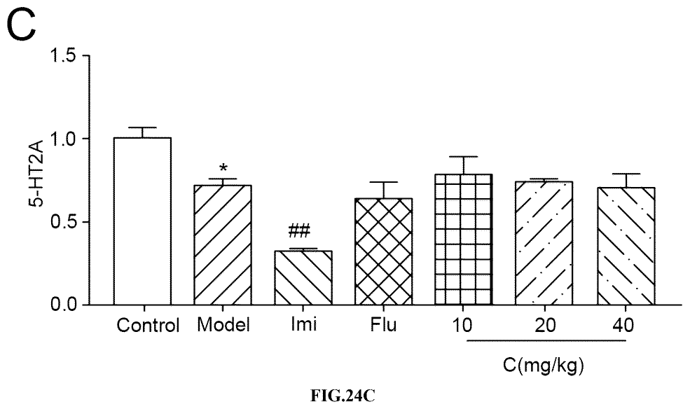

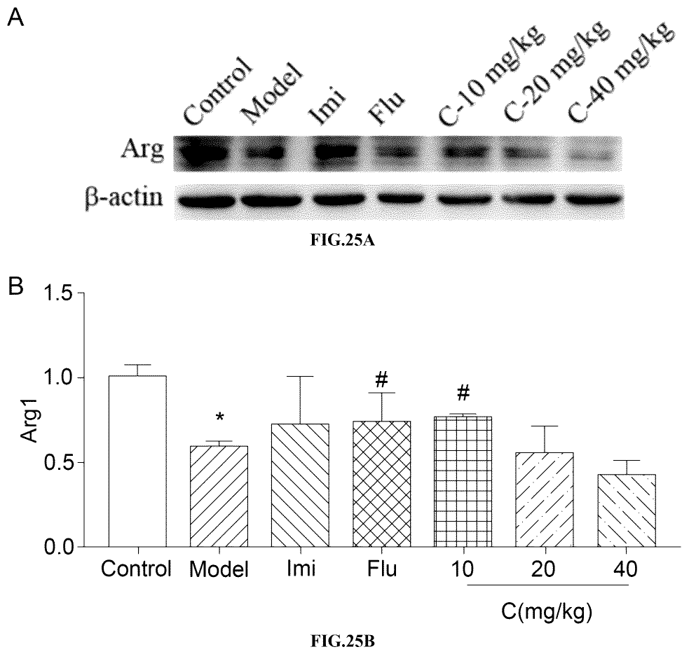

Cluster analysis of ions at 100 Da to 2,000 Da showed that the normal group and the low-dose group were clustered together, while the high-dose group and the medium-dose group were clustered together, indicating that the low-dose group of the compound could better promote the recovery of mice to normal (FIG. 22).
(6) After 21 d of drug administration, the expression of CREB protein, 5-HT1A protein, and Arg1 protein in the hippocampus of mice in each treatment group was statistically analyzed. The results showed that compared with the normal group, the CREB protein level in the model group was significantly reduced, while the 10 mg/kg BEL treatment group could significantly reverse this phenomenon. There was no significant difference in the expression of p-CREB protein among the groups. Compared with the normal group, the phosphorylation level of CREB in the model group was significantly increased; and compared with the model group, the phosphorylation level of CREB in the 40 mg/kg BEL treatment group was significantly decreased (FIGS. 23(A-D)), indicating that BEL could achieve antidepressant effects by regulating the expression of CREB protein in the mouse hippocampus. Compared with the normal group, the expression of 5-HT1A protein in the hippocampus of mice in the model group was significantly decreased; and compared with the model group, the 10 and 20 mg/kg BEL treatment groups could significantly promote the expression of 5-HT1A protein in the hippocampus of mice. Compared with the normal group, the expression of 5-HT2A protein in the hippocampus of mice in the model group was significantly decreased; and compared with the model group, the BEL treatment group had no effect on the expression of 5-HT2A in the hippocampus of mice (FIGS. 24(A-C)), indicating that BEL could achieve antidepressant effects by regulating the expression of 5-HT1A protein in the hippocampus of mice. Compared with the normal group, the expression of Arg1 protein, a marker of M2 polarization, in the hippocampus of mice in the model group was significantly reduced. Compared with the model group, 10 mg/kg BEL could significantly reverse the decrease in Arg1 expression (FIGS. 25(A-B)), indicating that BEL could achieve antidepressant effects by promoting the polarization of microglia to M2.

### Test Example 4

### In Vitro Antidepressant Activity Test of BEL-Containing Extract

1. Construction of HT22 cell model

T22 cells were randomly divided into a control group, a BEL-containing extract low-dose group (C-TQWL), a BEL-containing extract medium-dose group (C-TQWM), and a BEL-containing extract high-dose group (C-TQWH).

Control group: HT22 cells were induced with 250 μM CORT.

BEL-containing extract group (C-TQWL): HT22 cells were induced with 250 μM CORT and the BEL-containing extract obtained in step 1 of Example 5, where the BEL-containing extract had a final concentration of 5 μg/mL.

BEL-containing extract group (C-TQWM): HT22 cells were induced with 250 M CORT and the BEL-containing extract obtained in step 1 of Example 5, where the BEL-containing extract had a final concentration of 10 μg/mL.

BEL-containing extract group (C-TQWH): HT22 cells were induced with 250 M CORT and the BEL-containing extract obtained in step 1 of Example 5, where the BEL-containing extract had a final concentration of 15 μg/mL.

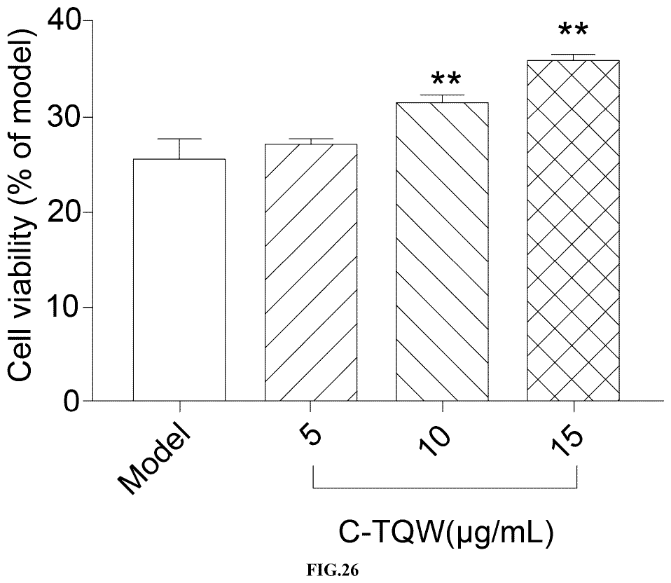

After 24 h of induction in each treatment group, the HT22 cell viability of each treatment group was counted. The results were shown in Table 7 and FIG. 26.

TABLE 7
HT22 cell viability induced by CORT in different
treatment groups (x̅ ± SD)

| | BEL-containing extract (μg/mL) | | |
|---|---|---|---|
| Control group | 5 | 10 | 15 |
| 27.54 ± 3.78 | 29.52 ± 1.36 | 31.08 ± 2.05* | 36.77 ± 1.21* |

NOTE:
compared with the control group,
*P < 0.01.

As shown in Table 7 and FIG. 26, the BEL-containing extract at the concentrations of 10 μg/mL and 15 μg/mL could significantly promote the proliferation of HT22 cells induced by CORT.

### Test Example 5

### In Vivo Antidepressant Effect Study of BEL-Containing Extract

After one week of adaptive feeding of experimental mice, 10 mice were randomly selected from 60 mice as a normal group, and the mice in the normal group were not treated in any way. The remaining mice were used as a modeling group, and the mice in the modeling group were induced with CORT for 21 d, with a CORT dose of 20 mg/kg and a dosing volume of 0.1 mL/20 mg. The dosing volume was changed once a week according to the weight of the mice, and the mice were subcutaneously administered in the groin of hind limb between 9:00 and 11:00 am every day. During the modeling, the weight, diet, and water intake of the mice were recorded, and the relevant data were counted once a week until the modeling was completed. During the modeling, the mice were subjected to behavioral analysis. 4 behavioral test designs were designed: FST, TST, SPT, and NSFT. Behavioral tests were conducted before the model was induced, and behavioral experiments were conducted 1 time one week after the stimulation began; the body weight was measured once a week, and the drug was administered for a total of 3 weeks. There were 5 mice/cage in the normal group and 5 mice/cage in the modeling group.

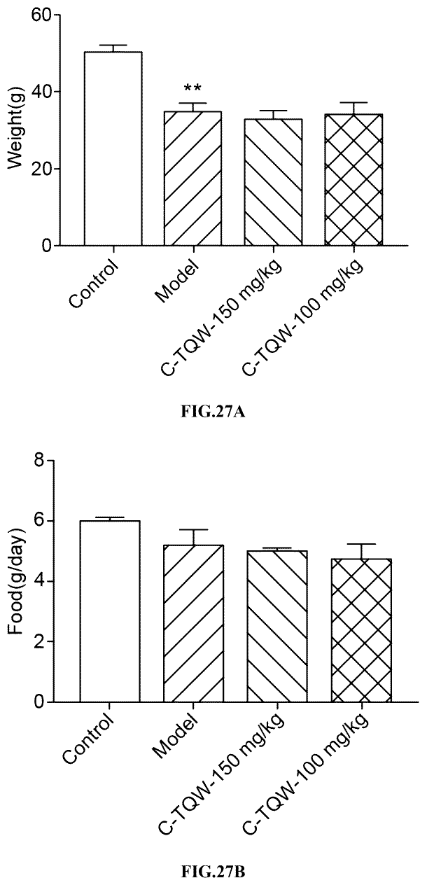

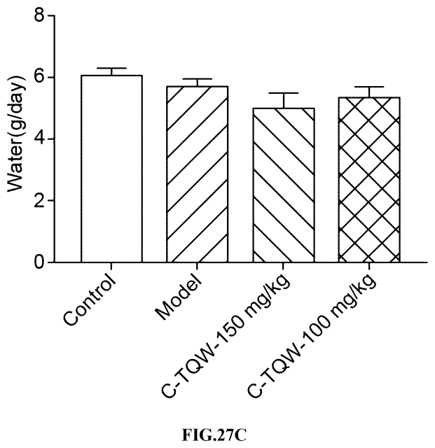

After the model induction was completed, 30 mice with successful modeling were screened by behavioral indicators and divided into: a model group, a BEL-containing extract high-dose (C-TQWH) group (150 mg/kg/d, gavage), and a BEL-containing extract low-dose (C-TQWL) group (100 mg/kg/d, gavage), where the BEL-containing extract was the BEL-containing extract obtained in step 1 of Example 5. The normal group received no treatment, the model group was gavaged with normal saline (0.9%) containing 1% DMSO+2% Tween 80, the BEL-containing extract high-dose group was gavaged with 150 mg/kg/d, and the BEL-containing extract low-dose group was gavaged with 100 mg/kg/d. Except for the mice in normal group, the other mice were gavaged at 9:00 to 11:00 every morning, and injected with CORT subcutaneously 2 h after the end of gavage for a total of 21 d. During the administration, body weight, diet, and water intake were recorded weekly, and the administration volume was changed once a week. Behavioral experiments were conducted once a week, and the serum 5-HT and CORT levels of mice were recorded. The results were shown in Tables 8 to 10 and FIG. 27A to FIG. 29B.

TABLE 8
Body weight, diet, and water intake of mice in each
treatment group (x̅ ± SD)

| Group | Body weight (g) | Diet (g/d) | Water intake (g/d) |
|---|---|---|---|
| Control | 46.72 ± 2.28 | 5.62 ± 0.17 | 6.17 ± 0.18 |
| Model | 33.08 ± 1.88# | 4.34 ± 0.52 | 5.70 ± 0.22 |
| C-TQWL | 34.73 ± 4.06 | 4.08 ± 0.97 | 5.21 ± 0.25 |
| C-TQWH | 32.65 ± 3.22 | 4.15 ± 0.04 | 4.66 ± 0.36 |

NOTE:
compared with the control group,
#P < 0.01, compared with the model group,
*P < 0.05.

As shown in Table 8 and FIGS. 27(A-C), BEL-containing extract had no significant effect on the body weight, diet, and water intake of mice. Compared with the normal group, the body weight of the model group decreased significantly; compared with the model group, high and low doses of BEL-containing extract had no significant effect on the body weight of mice. Compared with the normal group, there was no statistically significant change in diet and water intake in the model group; compared with the model group, high and low doses of BEL-containing extract had no significant effect on the diet and water intake of mice.

TABLE 9
Results of behavioral test of mice in each treatment group (x̅ ± SD)

| Group | FST (s) | TST (s) | SPT (%) | NSFT (s) |
|---|---|---|---|---|
| Control | 197.44 ± 43.33 | 94.25 ± 16.74 | 16.29 ± 1.36 | 57.38 ± 22.19 |
| Model | 212.00 ± 16.81# | 155.83 ± 33.53# | 9.98 ± 0.87# | 107.50 ± 22.55# |
| C-TQWL | 180.63 ± 25.70 | 97.50 ± 47.41 | 8.28 ± 0.86 | 93.43 ± 35.89 |
| C-TQWH | 157.50 ± 40.36* | 80.83 ± 35.41** | 12.82 ± 0.59 | 61.13 ± 31.28 |

NOTE:
compared with the control group,
#P < 0.05, compared with the model group,
*P < 0.05.

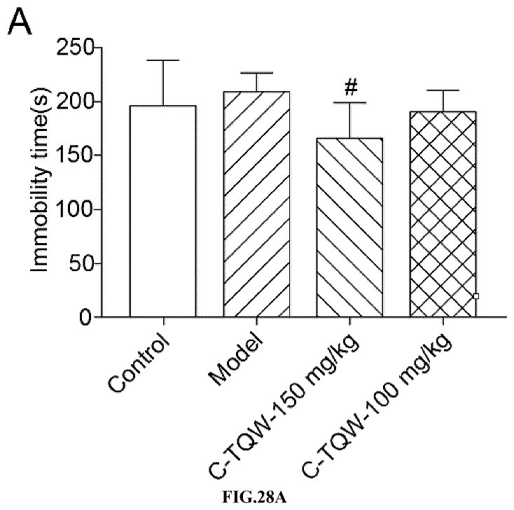

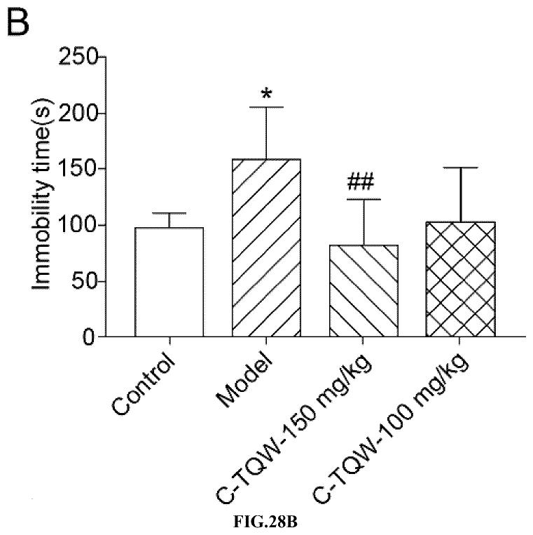

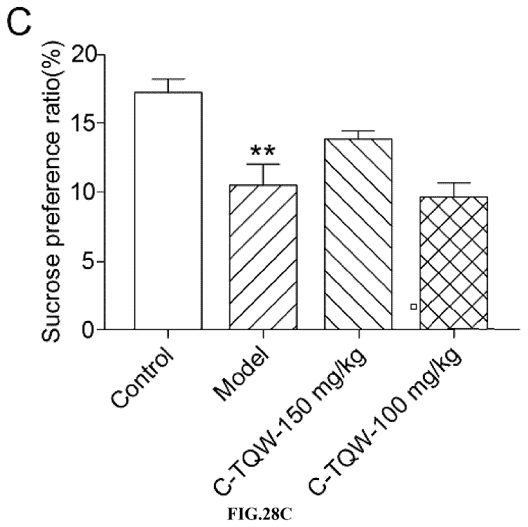

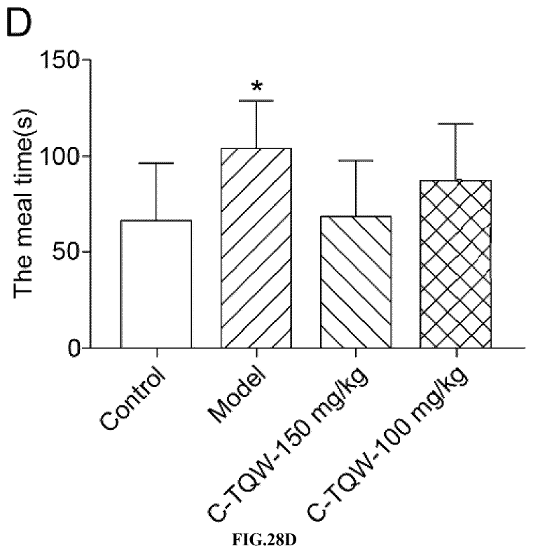

As shown in Table 9 and FIGS. 28(A-D), compared with the normal group, the model group showed obvious depressive-like behavior, which was manifested by a significant increase in the immobility time in the FST and TST tests, a decrease in the preference for sugar water in the SPT test, and a significant increase in the start time of eating in mice in the NSFT test. Compared with the model group, high dose of BEL-containing extract significantly reduced the immobility time of mice in FST and TST, but had no improving effect on the reduced sugar water preference and prolonged start time of eating in mice. Low dose of BEL-containing extract had no ameliorative effect on the depressive-like behavior of mice.

TABLE 10
Serum 5-HT and CORT levels of mice in each treatment
group (x̅ ± SD)

| Group | 5-HT (ng/mL) | CORT (ng/mL) |
|---|---|---|
| Control | 155.56 ± 29.70 | 119.63 ± 6.47 |
| Model | 98.47 ± 14.40# | 195.47 ± 18.51# |
| C-TQWL | 122.09 ± 11.86 | 157.50 ± 3.96 |
| C-TQWH | 149.08 ± 12.59* | 131.54 ± 5.88* |

NOTE:
compared with the control group,
#P < 0.05, compared with the model group,
*P < 0.05.

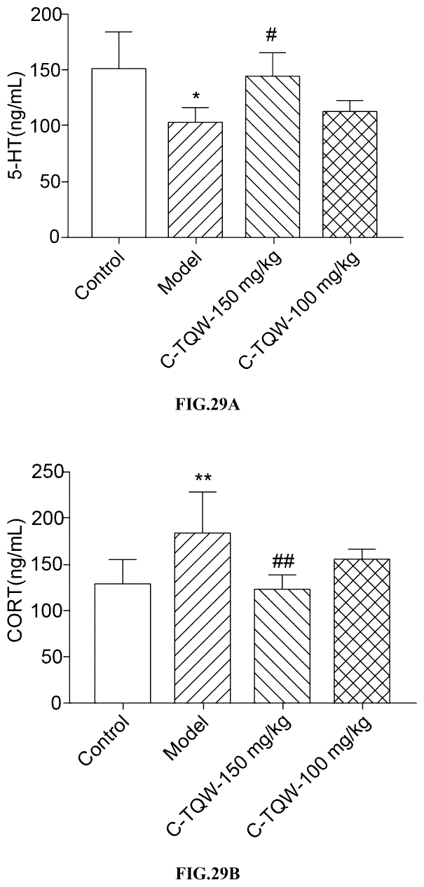

As shown in Table 10 and FIGS. 29(A-B), compared with the normal group, the serum 5-HT level in the model group was significantly reduced; compared with the model group, the high dose of BEL-containing extract could significantly increase the serum 5-HT level. Compared with the normal group, the serum CORT level in the model group was significantly increased; compared with the model group, the high dose of BEL-containing extract could significantly reduce the serum CORT level of mice. Low dose of BEL-containing extract had no significant regulatory effect on the serum 5-HT and CORT levels of mice.

### Example 7

### A Solid Beverage for Improving Depression

20 wt. % maltodextrin, 0.3 wt. % sodium carboxymethyl cellulose, and 5 wt. % sucrose or xylitol were added into the BEL prepared in Example 1, mixed with an SYH laboratory three-dimensional motion mixer, and granulated in a conventional manner to obtain the solid beverage.

### Example 8

### A Candy Tablet Health Food for Improving Depression

20 wt. % maltitol or xylitol or aspartame or steviol glycoside, 15 wt. % microcrystalline cellulose, and 5 wt. % magnesium stearate were added into the BEL-containing extract prepared in Example 2, mixed with an SYH laboratory three-dimensional motion mixer, dry granulated, and then tableted with a ten-punch tablet press (ZPSX) (pressure 40 KN to 60 KN) to obtain the candy tablet.

### Example 9

### A Capsule Drug for Treating Depression

(30-50) wt. % lactose or pregelatinized starch, 2 wt. % magnesium stearate, and 5 wt. % talc or aerosil were added into the BEL obtained in Example 3, mixed with an SYH laboratory three-dimensional motion mixer, dry granulated, and then filled with an NJP-2-200C fully automatic capsule filling machine to obtain the capsule.

### Example 10

### A Microcapsule Drug for Treating Depression

The BEL prepared in Example 4 was mixed with sodium alginate at a ratio of 1:2 g/g, uniformly dispersed in water, such that a concentration of sodium alginate was 2% to obtain a solution A of the BEL and sodium alginate; a chitosan acetic acid solution with a chitosan mass concentration of 1.0% was prepared, added with a certain amount of CaCl₂ solution to make a mass concentration 1.8%, and a pH value was adjusted to 5.5 with a certain concentration of alkaline solution, and stirred evenly to obtain a solution B; the solution A of the BEL and sodium alginate was sprayed into the solution B at a particle diameter of 150 μm using a microcapsule granulator, a film-forming reaction was conducted by stirring for 20 min, and microspheres were collected, washed, and freeze-dried to obtain the microcapsule.

### Example 11

### A Dripping Pill Drug for Treating Depression

55 wt. % of a medicinal matrix PEG-3350 and 40 wt. % of a medicinal matrix PEG-8000 were added into the BEL prepared in Example 5, completely melted in a water bath at 85° C., stirred and mixed well, poured into the loading tank of a small pill dropping machine, stirred and kept warm for 30 min, and dripped into dimethyl silicone oil coolant at 30 drops/min at a dripping distance of 10 cm. After the dripping pills were completely shrunk and condensed into shape, they were taken out, and the coolant on a surface of the dripping pills was removed by an oil thrower to obtain the dripping pill drug.

Based on the above contents, it can be seen that the BEL or BEL-containing extract as an active ingredient can significantly improve a depressive behavior of mice, regulate concentrations of 5-HT and CORT, protect nerve cells, and improve synaptic plasticity. Moreover, the BEL or BEL-containing extract can penetrate the BBB and has a significant effect on improving and treating depression.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for preparing an antidepressant product, comprising adding bellidifolin (BEL) and/or a BEL-containing extract to the antidepressant product.

2. The method according to claim 1, wherein the antidepressant product comprises 0.1% to 99.9% of the BEL by weight.

3. The method according to claim 1, wherein the antidepressant product is selected from the group consisting of a drug, a health product, and a functional food.

4. The method according to claim 1, wherein the BEL-containing extract comprises 1.0% to 95.0% of the BEL by weight.

5. The method according to claim 1, comprising preparing the BEL-containing extract, wherein the preparing of the BEL-containing extract comprises the following steps:
mixing a BEL-containing raw material and an ethanol solution with a volume concentration of 65% to 75% to allow heating reflux extraction, and separating ethanol from an obtained extract to obtain a BEL crude extract;
dissolving the BEL crude extract in hot water at not less than 90° C., and mixing an obtained solution with concentrated hydrochloric acid to allow hydrolysis to obtain an acid hydrolyzate;
adjusting a pH value of the acid hydrolyzate to 7 to allow salting out, and collecting an obtained first precipitate;
dissolving the first precipitate by heating in an ethanol solution with a volume concentration of 95.0% or pure methanol, cooling, conducting solid-liquid separation, and collecting an obtained solution; and
mixing the solution with water, allowing an obtained mixed solution to stand, and collecting an obtained second precipitate to obtain the BEL-containing extract.

6. The method according to claim 5, wherein the heating reflux extraction is conducted at not less than 90° C. for 1 to 3 times, each time for 2 h.

7. The method according to claim 5, wherein a solid-to-liquid ratio of the BEL-containing raw material and the ethanol solution are 1 g: 20 mL to 1 g:30 mL during each time of the heating reflux extraction; and
the BEL-containing raw material is selected from the group consisting of Swertia mussotii, Swertia franchetiana, Swertia chirayita, Lomatogonium carinthiacum, and Halenia elliptica.

8. The method according to claim 5, wherein the hydrolysis is conducted at not less than 90° C. for 0.5 h to 1 h.

9. The method according to claim 5, wherein the pH value of the acid hydrolyzate is adjusted using a sodium hydroxide solution;
the salting out is conducted with sodium chloride for not less than 2 h; and
the mixed solution is allowed to stand for not less than 24 h.

10. The method according to claim 5, wherein after obtaining the BEL-containing extract, further purifying the BEL-containing extract by preparative liquid chromatography to obtain BEL with a purity of 95.0% to 99.9%.

11. The method according to claim 3, wherein the antidepressant product comprises 0.1% to 99.9% of the BEL by weight.

12. The method according to claim 10, wherein the heating reflux extraction is conducted at not less than 90° C. for 1 to 3 times, each time for 2 h.

13. The method according to claim 10, wherein a solid-to-liquid ratio of the BEL-containing raw material and the ethanol solution are 1 g: 20 mL to 1 g:30 mL during each time of the heating reflux extraction; and
the BEL-containing raw material is selected from the group consisting of Swertia mussotii, Swertia franchetiana, Swertia chirayita, Lomatogonium carinthiacum, and Halenia elliptica.

14. The method according to claim 10, wherein the hydrolysis is conducted at not less than 90° C. for 0.5 h to 1 h.

15. The method according to claim 10, wherein the pH value of the acid hydrolyzate is adjusted using a sodium hydroxide solution;
the salting out is conducted with sodium chloride for not less than 2 h; and
the mixed solution is allowed to stand for not less than 24 h.
